SEQUENCE LISTING (34 sequences)

SEQ ID NO: 1 - DNA, 2114 bp, Pseudomonas aeruginosa
  CDS (329)...(2086): ybtq
acgatggtccgtgggtagtcctgcgcctgtgcggcggcggaccccacggcaaagaacagcgtcaggagtgatcgagcggtacgcatggaggccttccttcccaggattcatatcgaccttcagtgtttcccgttgcctgagaaaaccgctccccgaatcgggaatcagttcgctcgcgatcgccttcctttctgggtagccgaaacttcttacccgctcataagctcggtccgtcccttcacatgacgaatctccgatgggtaatgacctaagtcgtatgggcgcgctgcggatactgctggagttgtcggtgggccaccgtttggccatggcaggagccttggctttgacccttttggctgtactggcggaactggcgccattcgccatcctgtacttcgctgtcgaggccctactcaggacgcctcaagccttcgcccaggaacttctgactctggctccctggctggtgggtggcatagtgctgaagtacatggcctatggcgtcgcctacctgatcagccatcacgctgcctacgcaatcatggcagtacgcgcagccgcctggcggccaagctcgatgatgcgcccttgcactggatacatgcacaggggtcgggcgcgcagaaacagtccgttattcaagaatgtcgagcgcatggaggcattcattgcccatcacaccgtggaggtcgccgccgcggtattggcgccactgtgtgtcacgacagccttgctctgggtggactggcggctggcaatggcggcgctggcagtcggtcccttggccctgctcgcttcaacattcgccatgcgcggagtgggtcaaaaccaagatcggttcaaccgggcaacggccagcctgaacaacgtgaccgttgagtacctgcgcaacatgccggtgctcaaggtgttcagtcgcagtgcctccgggttccggctgctgcgacggcagcttcatgcctactatcgactaactgatcagatcactcgcaacaccgttccagggtgggcgctgttcaccagcgtcctgggtgcccacctgctcctgttgcttccggtcggcgcatggcttcatgcgcgtggggaaatcggtgttgctcaagtggtggtggcagtgctgctgggggctgggatttttcgtcccttgctaaaagtcagtcgcttcatcatggacataccgccgattctcgcaggcctacgcaggatggcaccgatactggcgctcagtcgcaagcgggggcgagcagatctgccggtggctgccaccgtacgggtcgacctcgaccaggtctgctttcgctatggcggacgccacgtgctgaccggggtgagcctgtccctggcgtcgggcaccttcaatgtactcctggggccatcggggtccggtaagtccaccatcgcgcaactgattgcgggcttactggcgccagagtcggggtcggtcacgatcaacgggaagtccatcgccaccctcagcgatgaggagcgtacccgctgcatagctctcgcggcccaggacgtgtttctcttcagcagggaacggtgcgcgacaacctggtgctccgcgcgcccgcaggcatccgaggcggagatctgccggcccgtgcgtgtggcgcaggcgcaggcgcttatcgaggggcttcctgccgggctacgacacccccgctcaatgaactgcacccgcttatctggcggcgagcgccagcgcctggcggtcgcacgggcattgcttgccgacgcggcggttctggtgctcgacgaatccgcggcgttcgccgacagcctgacgcaacgcgcgttctttcaggcgctgctcgaggagtacccagagaaaacactgcttgtcgttgcacacaggctgcatggaatcgagcaagccgaccagatactagtgcttgaggaaggtgcattgtccttgtgcggacgccatgaccaactcatgagcgagagcgattactaccggtccatgtggatgcatgaggagttcgccgagcgctggagcttgcgcggcgctgcgccgtcccaagacaggacgcaagagtcggcagcattgccggcgacatctaccgcagggggagattgagacatggaggcggtcacctcttggacaa

SEQ ID NO: 2 - protein, 585 aa, Pseudomonas aeruginosa
MAGALALTLLAVLAELAPFAILYFAVEALLRTPQAFAQELLTLAPWLVGGIVLKYMAYGVAYLISHHAAYAIMAVRAAAWRPSSMMRPCTGYMHRGRARRNSPLFKNVERMEAFIAHHTVEVAAAVLAPLCVTTALLWVDWRLAMAALAVGPLALLASTFAMRGVGQNQDRFNRATASLNNVTVEYLRNMPVLKVFSRSASGFRLLRRQLHAYYRLTDQITRNTVPGWALFTSVLGAHLLLLLPVGAWLHARGEIGVAQVVVAVLLGAGIFRPLLKVSRFIMDIPPILAGLRRMAPILALSRKRGRADLPVAATVRVDLDQVCFRYGGRHVLTGVSLSLASGTFNVLLGPSGSGKSTIAQLIAGLLAPESGSVTINGKSIATLSDEERTRCIALAAQDVFLFSRERCATTWCSARPQASEAEICRPVRVAQAQALIEGLPAGLRHPRSMNCTRLSGGERQRLAVARALLADAAVLVLDESAAFADSLTQRAFFQALLEEYPEKTLLVVAHRLHGIEQADQILVLEEGALSLCGRHDQLMSESDYYRSMWMHEEFAERWSLRGAAPSQDRTQESAALPATSTAGGD

SEQ ID NO: 3 - DNA, 1030 bp, Pseudomonas aeruginosa
  CDS (470)...(1009): pila
gatccttgggcagactgtcccgctcaaggctgttcaggtcgcagtaggcgataccgaattgatccgcagccagttcgatcaaggcctgccctttcactagcttgctctgtaccagataggttaccagcgacaacttgttgcgctgggcctgcgattgggcctgttgggcggccttttcatcgagaagttcatgcaggacgagctggcgagccaagccgctgagttgaattgtgtcgttcattgggagtggtcgcataaggctagatatgctgccttataacgcagaagaggagggctgccaaaccgagaaggtcggactgccgaaaagtgtcacatattgtcggtttgtggggctgccgctggaggggagaggctgcaccggcttcgtagctatggagttttcaggttggcatgcaagatgctttatggtcgtcaggcgttaggcctggatatatcaatggagagatacatgacagctcagaaaggctttaccttgatcgaactgatgatcgtggttgcgatcatcggtattcttgctgccgtcgctttgccggcatatcaggattacaccattcgtgctcgcgtgacagagggggttggcctggctgccagcgccaagacgcttattggcgatagctctgccactgccggtgagctagccgcttcggcaagggtctggaatgctcaagccggtaacgccggtgctaccagtaagtatgtgacctctgtacaaattgcagaggcgactggtgaaatcactgttactttcaatgccgcaaacgtgggtaatattccggctaactctaccctggtatttactccctatgtgcagaatgctgccggtgccccgactcaattgggtgccagttatgcttccggtgtgactggctctattgactggggttgtgcatcggactccaacgcggtttccagtggtaccgaccgcaatatgcctgccctgactgcaggtaccctgccggctcgtttcgctcctagcgaatgccgctaataggctaatgctgaaaagatc

SEQ ID NO: 4 - protein, 179 aa, Pseudomonas aeruginosa
MTAQKGFTLIELMIVVAIIGILAAVALPAYQDYTIRARVTEGVGLAASAKTLIGDSSATAGELAASARVWNAQAGNAGATSKYVTSVQIAEATGEITVTFNAANVGNIPANSTLVFTPYVQNAAGAPTQLGASYASGVTGSIDWGCASDSNAVSSGTDRNMPALTAGTLPARFAPSECR

SEQ ID NO: 5 - DNA, 1160 bp, Pseudomonas aeruginosa
  CDS (47)...(1160): pilc
gatcaccagcctggaggaagtcaaccgcgtgaccaaggactaatccatggcggacaaagcgttaaagaccagtgtcttcatctgggaaggcactgataagaaaggcagcaaggtcaaaggagagttggccgggcaaaaccctatgctggtgaaggctcaactgcgcaaacagggtatcaacccactaaaggtccgcaagaaaggtatcaccctgtgggcagggaagaagattaagcccatggacatcgccttgtttcactcggcagatgtctaccatgatgggtgccggcgaccggtactgcaatcttttgacatcatcggcgaaggattcgaaaatccaaacatgcgcaagctagtcgatgagatcaagcaggatgttgccgccggtaacagcttagccagttcacttcgaaagaaacccatttacttcgatgatctctactgcaacctggtcgatgctggcgaacagtccggtgctttggagacattattggatcgggtagcaacttataaagaaaagacagaatccctgaaagccaaaattaaaaaagccatgacttatcccattgcagtaattgtagtggcccttgtagtatcggcgatccttctgataaaagtggtcccacagttccagtccgtatttgcaaattttggtgccgagttgccggcctttactcaaatggtcatcaatctttccgagatgcttcaagagtggtggctcatagtgcttattggtctttttgccgcagcttttgcatttagggaagctcatcatttgggatcagtagatcggggcctgctgaaactacctatcatcggcgggatactttacaaatcagctatcgcccgctacgcccgaacgctatccactacctttgcggctggagtgcctctggtagaagctctggactccgtttccggagcaactggcaacgtggtattcaagaatgccgttaccaagatcaaacaagacgtgtccagcggcatgcaattgaacttttccatgcgcaccaccaatgttttccccagtatggctatcagatggctgcctggcgaggaatcaggttcgctagatgagatgttaggaaaagttgccggtttctatgaggaagaagtcgataacgccgtcgacaacctgacaacgttgatggagcgtgatc

SEQ ID NO: 6 - protein, 371 aa, Pseudomonas aeruginosa
MADKALKTSVFIWEGTDKKGSKVKGELAGQNPMLVKAQLRKQGINPLKVRKKGITLWAGKKIKPMDIALFHSADVYHDGCRRPVLQSFDIIGEGFENPNMRKLVDEIKQDVAAGNSLASSLRKKPIYFDDLYCNLVDAGEQSGALETLLDRVATYKEKTESLKAKIKKAMTYPIAVIVVALVVSAILLIKVVPQFQSVFANFGAELPAFTQMVINLSEMLQEWWLIVLIGLFAAAFAFREAHHLGSVDRGLLKLPIIGGILYKSAIARYARTLSTTFAAGVPLVEALDSVSGATGNVVFKNAVTKIKQDVSSGMQLNFSMRTTNVFPSMAIRWLPGEESGSLDEMLGKVAGFYEEEVDNAVDNLTTLMERD

SEQ ID NO: 7 - DNA, 1150 bp, Pseudomonas aeruginosa
  CDS (125)...(1150): uvrD
gatccacgcatgcattgtagcgacgcctccgcagcccttcgtggttcgtgctggcgcaggttccggcaagaccacctccctcatcaaggcgctggactgggtgatctcggagcacggcgccagcatgcgggcgaggaagcagatagtcgcgtgcatcacgtataccgaccttgccaccaatgaaatcctggcggacgtcaacgatgacccgctggttcatgtctcgaccatccacagcttttactggtctattgcaaagacgttccaggccgacatcaaggtttggctgcagaacgacatccgcaggcggatctccgaacttgaagaagagttcgagaattacagctcgcgtgtccggcagaccacgcgcgacaggaacaaggccgaccaagagcgatatgtccgaagcctggaggctgtggccggcgtcaggacgttcaactacggcgtgggcagtgactacgccaagggcatacttggccacgaggacatccttcagctcgccgacttcctgctacaaaaccgcccgctgttccgacgggtcgtggcgctgagctacccgttcgtgtttatcgatgagagtcaggacacgttcccgggtgtagtgaagtctttcaaggaagtggaagcccagatgcagggcaagttctgccttggttttttcggcgacccgatgcagtcgatcttcatgagaggcgcaggggacatccagcttgaggatcattggcgggccatcacgaagccggagaactttcgctgcgccaagcagatccttgacgtcgccaatgccgtgcgcgcgcagggcgatggcatggagcaagtccgcgggctgcacgagagggtcgatgggaacctcaagctggtggaggggtcggcccggatgttcgtcttgccgaacacgctgaaccgaaccgaggctttggcaagagtccgagcgtggagctcggcgacgaacaacgacgagggttggacaaccccagacatcgcagtcaagattcttgtcatcgtgcaccgcatggccgcaaaccggcttggcttcggcggcatctactcggcgctgaacgacaagacgtcggatgccatgaagcaagggatgcaggacggcaccggttggcccgttcgacccttcctaagttttgcgctaccgatc

SEQ ID NO: 8 - protein, 342 aa, Pseudomonas aeruginosa
MRARKQIVACITYTDLATNEILADVNDDPLVHVSTIHSFYWSIAKTFQADIKVWLQNDIRRRISELEEEFENYSSRVRQTTRDRNKADQERYVRSLEAVAGVRTFNYGVGSDYAKGILGHEDILQLADFLLQNRPLFRRVVALSYPFVFIDESQDTFPGVVKSFKEVEAQMQGKFCLGFFGDPMQSIFMRGAGDIQLEDHWRAITKPENFRCAKQILDVANAVRAQGDGMEQVRGLHERVDGNLKLVEGSARMFVLPNTLNRTEALARVRAWSSATNNDEGWTTPDIAVKILVIVHRMAANRLGFGGIYSALNDKTSDAMKQGMQDGTGWPVRPFLSFALPI

SEQ ID NO: 9 - DNA, 24 bp, Pseudomonas aeruginosa
agcactctccagcctctcaccgca

SEQ ID NO: 10 - DNA, 12 bp, Pseudomonas aeruginosa
gatctgcggtga

SEQ ID NO: 11 - DNA, 24 bp, Pseudomonas aeruginosa
accgacgtcgactatccatgaaca

SEQ ID NO: 12 - DNA, 12 bp, Pseudomonas aeruginosa
gatctgttcatg

SEQ ID NO: 13 - DNA, 19 bp, Pseudomonas aeruginosa
catttagggaagctcatca

SEQ ID NO: 14 - DNA, 22 bp, Pseudomonas aeruginosa
gaactgtgggaccacttttatc

SEQ ID NO: 15 - DNA, 19 bp, Pseudomonas aeruginosa
ctagtgaaagggcaggcct

SEQ ID NO: 16 - DNA, 18 bp, Pseudomonas aeruginosa
ggcatgcaagatgcttta

SEQ ID NO: 17 - DNA, 19 bp, Pseudomonas aeruginosa
actcttcttcaagttcgga

SEQ ID NO: 18 - DNA, 19 bp, Pseudomonas aeruginosa
cagatgcagggcaagttct

SEQ ID NO: 19 - DNA, 19 bp, Pseudomonas aeruginosa
tgatgagcttccctaaatg

SEQ ID NO: 20 - DNA, 19 bp, Pseudomonas aeruginosa
actggacatagggggtaag

SEQ ID NO: 21 - DNA, 20 bp, Pseudomonas aeruginosa
cgtcgttccagtttcctctc

SEQ ID NO: 22 - DNA, 22 bp, Pseudomonas aeruginosa
tgagcttccctaaatgcaaaag

SEQ ID NO: 23 - DNA, 17 bp, Pseudomonas aeruginosa
tgaagcatctcggaaag

SEQ ID NO: 24 - DNA, 18 bp, Pseudomonas aeruginosa
gaaggatcgccgatacta

SEQ ID NO: 25 - DNA, 17 bp, Pseudomonas aeruginosa
gattgcagtaccggtcg

SEQ ID NO: 26 - DNA, 20 bp, Pseudomonas aeruginosa
tccttctgataaaagtggtc

SEQ ID NO: 27 - DNA, 20 bp, Pseudomonas aeruginosa
gcagcaaggtcaaaggagag

SEQ ID NO: 28 - DNA, 22 bp, Pseudomonas aeruginosa
tgagcttccctaaatgcaaaag

SEQ ID NO: 29 - DNA, 18 bp, Pseudomonas aeruginosa
gaaaggctttaccttgat

SEQ ID NO: 30 - DNA, 17 bp, Pseudomonas aeruginosa
aggagcgaaacgagccg

SEQ ID NO: 31 - DNA, 19 bp, Pseudomonas aeruginosa
ctacgcaatcatggcagta

SEQ ID NO: 32 - DNA, 20 bp, Pseudomonas aeruginosa
cgattccatgcagcctgtgt

SEQ ID NO: 33 - DNA, 19 bp, Pseudomonas aeruginosa
cacgcatgcattgtagcga

SEQ ID NO: 34 - DNA, 18 bp, Pseudomonas aeruginosa
gatcggtagcgcaaaact

(19) United States
(12) Patent Application Publication
(10) Pub. No.: US 2005/0089988 A1
(43) Pub. Date: Apr. 28, 2005

(54) Pseudomonas virulence factors and uses thereof

(76) Inventors: Stephen B. Calderwood, Wellesley, MA (US); Ji Young Choi, Seon Gnam-Si (KR); Costi D. Sifri, Charlottesville, VA (US); Boyan C. Goummerv, Randolph, MA (US); Laurence G. Rahme, Brookline, MA (US); Frederic M. Ausubel, Newton, MA (US)

Correspondence Address: CLARK & ELBING LLP, 101 FEDERAL STREET, BOSTON, MA, 02110, US

(21) Appl. No.: 10/497,846

(22) PCT Filed: Jan. 3, 2003
(86) PCT No.: PCT/US03/00184

Related U.S. Application Data
(60) Provisional application No. 60/345,287, filed on Jan. 4, 2002.

(51) Int. Cl. A61K 39/02; C12N 1/21; C12N 15/74

(52) U.S. Cl. 435/252.34; 424/200.1; 530/350; 435/69.3; 435/320.1

(57) ABSTRACT

Disclosed are bacterial virulence polypeptides and nucleic acid sequences (e.g., DNA) encoding such polypeptides, and methods for producing such polypeptides by recombinant techniques. Also provided are methods for utilizing such polypeptides to screen for antibacterial or bacteriostatic compounds.

### BACKGROUND OF THE INVENTION

The invention features nucleic acid molecules associated with virulence of a pathogen, methods for isolating such molecules, and the use of such molecules in human, agricultural, and veterinary practice. The invention also features polypeptides encoded by these nucleic acid molecules and uses thereof.

Pseudomonas aeruginosa is an opportunistic pathogen that frequently causes severe systemic infections, particularly in patients with cystic fibrosis, burns, or immunosuppression. A soil inhabitant, P. aeruginosa is widely distributed in the natural environment and can also act as a plant pathogen. Recently, Rahme et al. (Science 268:1899-1902, 1995) have exploited the broad host range of this pathogen and have shown that a clinical isolate of P. aeruginosa, strain PA14, is capable of causing disease in both an Arabidopsis thaliana leaf infiltration model and in a mouse full-thickness skin thermal burn model. Furthermore, mutations in a variety of PA14 genes reduced the virulence of this strain simultaneously for both plants and mice, suggesting that at least some of the mechanisms of pathogenesis of P. aeruginosa infection may be conserved in evolutionarily divergent hosts.

These results have subsequently been extended to show that P. aeruginosa can also act as a pathogen for a variety of additional non-vertebrate hosts, including Caenorhabditis elegans, (Mahajan-Miklos et al., Cell 96:47-56, 1999; Tan et al., Proc. Natl. Acad. Sci. U.S.A 96:715-720, 1999; Tan et al., Proc. Natl. Acad. Sci. U.S.A 96:2408-2413, 1999), Drosophila melanogaster (D'Argenio et al., J. Bacteriol. 183:1466-1471, 2001), and the greater wax moth Galleria mellonella (Jander et al., J Bacteriol 182:3843-3845, 2000). This has led to the development of a multihost pathogenesis system in which plants, nematodes, and insects have been used as adjuncts to animal models for the identification and study of bacterial virulence factors of P. aeruginosa. The relevance to mammalian pathogenesis of virulence factors identified using these screens has been confined using a mouse full-thickness burn model (Stevens et al., J. Burn Care Rehabil. 15:232-235, 1994). Remarkably, among twenty genes in P. aeruginosa strain PA14 that are required for pathogenesis in at least one of the three different invertebrate hosts (a plant, a nematode, or an insect), seventeen of the genes were also required for full pathogenicity in a mouse burn model.

Another approach to identifying virulence factors in bacteria is to take advantage of naturally occurring differences in pathogenicity between isolates of the same species, utilizing one of a variety of subtractive techniques to recover genes present in one isolate but not the other, such as those found on pathogenicity islands. One such technique is representational difference analysis “RDA,” a procedure involving subtractive hybridization and kinetic enrichment that has been used previously to recover differences between two complex genomes. Recently, RDA was adapted for use in detecting and cloning genomic differences between two closely related bacterial species or isolates of the same species (Calia et al., Infect. Immun. 66:849-852, 1998; Perrin et al., Infect. Immun. 67:6119-6129, 1999; Tinsley et al., Proc. Natl. Acad. Sci. U.S.A 93:11109-11114, 1996).

Opportunistic pathogens cause serious infections in many patients with compromised immune systems. As drug-resistant variants of these pathogens develop, new drugs are required to fight infection. A need for new drug-targets therefore exists in the art. This invention addresses that need by identifying virulence genes that may serve as targets for new antibacterial therapies.

### SUMMARY OF THE INVENTION

We have identified and characterized a number of nucleic acid molecules and polypeptides that are involved in conferring pathogenicity and virulence to a pathogen. This discovery therefore provides a basis for drug-screening assays aimed at evaluating and identifying “anti-virulence” agents which are capable of blocking pathogenicity and virulence of a pathogen, e.g., by selectively switching pathogen gene expression on or off, or which inactivate or inhibit the activity of a polypeptide that is involved in the pathogenicity of a microbe. Drugs that target these molecules are useful as such anti-virulence agents.

In one aspect, the invention features an isolated nucleic acid molecule including a sequence substantially identical to any one of ybtQ (SEQ ID NO:1), pilA (SEQ ID NO:3), pilC (SEQ ID NO:5), or uvrD (SEQ ID NO:7). Preferably, the isolated nucleic acid molecule includes any of the above-described sequences or a fragment thereof; and is derived from a pathogen (e.g., from a bacterial pathogen such as Pseudomonas aeruginosa PA14). Additionally, the invention includes a vector and a cell, each of which includes at least one of the isolated nucleic acid molecules of the invention; and a method of producing a recombinant polypeptide involving providing a cell transformed with a nucleic acid molecule of the invention positioned for expression in the cell, culturing the transformed cell under conditions for expressing the nucleic acid molecule, and isolating a recombinant polypeptide. The invention further features recombinant polypeptides produced by such expression of an isolated nucleic acid molecule of the invention, and substantially pure antibodies that specifically recognize and bind such recombinant polypeptides.

In another aspect, the invention features a substantially pure polypeptide including an amino acid sequence that is substantially identical to the amino acid sequence of any one of YbtQ (SEQ ID NO:2), pilA (SEQ ID NO:4), pilC (SEQ ID NO:6), and UvrD (SEQ ID NO:8). Preferably, the substantially pure polypeptide includes any one of the above-described sequences or a fragment thereof; and is derived from a pathogen (e.g., from a bacterial pathogen such as Pseudomonas aeruginosa PA14).

In yet another related aspect, the invention features a method for identifying a compound which is capable of decreasing the expression of a pathogenic virulence factor (e.g., at the transcriptional or post-transcriptional levels), involving (a) providing a pathogenic cell expressing any one of the isolated nucleic acid molecules of the invention; and (b) contacting the pathogenic cell with a candidate compound, where a decrease in expression of the nucleic acid molecule following contact with the candidate compound identifies a compound which decreases the expression of a pathogenic virulence factor. In preferred embodiments, the pathogenic cell infects a mammal (e.g., a human) or a plant.

In yet another related aspect, the invention features a method for identifying a compound which binds a polypeptide (e.g., YbtQ, PilA, PilC, or UvrD), involving (a) contacting a candidate compound with a substantially pure polypeptide including any one of the amino acid sequences of the invention under conditions that allow binding; and (b) detecting binding of the candidate compound to the polypeptide.

In addition, the invention features a method of treating a pathogenic infection in a mammal, involving (a) identifying a mammal having a pathogenic infection; and (b) administering to the mammal a therapeutically effective amount of a composition which inhibits the expression or activity of a polypeptide encoded by any one of the nucleic acid molecules of the invention. In preferred embodiments, the pathogen is Pseudomonas aeruginosa PA14.

In yet another aspect, the invention features a method of treating a pathogenic infection in a mammal, involving (a) identifying a mammal having a pathogenic infection; and (b) administering to the mammal a therapeutically effective amount of a composition which binds and inhibits a polypeptide encoded by any one of the amino acid sequences of the invention. In preferred embodiments, the pathogenic infection is caused by Pseudomonas aeruginosa PA14.

Moreover, the invention features a method of identifying a compound which inhibits the virulence of a Pseudomonas cell, involving (a) providing a Pseudomonas cell; (b) contacting the cell with a candidate compound; and (c) detecting the presence of a phenolate-thiazole siderophore, wherein a decrease in the phenolate-thiazole siderophore in a treated cell relative to an untreated cell is an indication that the compound inhibits the virulence of the Pseudomonas cell. In preferred embodiments, the cell is Pseudomonas aeruginosa (such as P. aeruginosa PA14); the cell is present in a cell culture; and the phenolate-thiazole siderophore is detected by spectroscopy.

By “isolated nucleic acid molecule” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

By “polypeptide” is meant any chain of amino acids, regardless of length or post-translational modification (for example, glycosylation or phosphorylation).

By an “isolated polypeptide” or a “substantially pur polypeptide” is meant a polypeptide of the invention that has been separated from components which naturally accompany it. Typically, the polypeptide is substantially pure when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source (for example, a pathogen); by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “substantially identical” is meant a polypeptide or nucleic acid molecule exhibiting at least 30% identity to a reference amino acid sequence (for example, any one of the amino acid sequences described herein) or nucleic acid sequence (for example, any one of the nucleic acid sequences described herein). Preferably, such a sequence is at least 30%, 35%, 40%, 45%, 50%, 55%, 60%, 65%, 70%, or 75%, more preferably 80% or 85%, and most preferably 90% or even 95%, 96%, 97%, 98%, or 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “transformed cell” is meant a cell into which (or into an ancestor of which) has been introduced, by means of recombinant DNA techniques, a DNA molecule encoding (as used herein) a polypeptide of the invention.

By “positioned for expression” is meant that the DNA molecule is positioned adjacent to a DNA sequence which directs transcription and translation of the sequence (i.e., facilitates the production of, for example, a recombinant polypeptide of the invention, or an RNA molecule).

By “purified antibody” is meant antibody which is at least 60%, by weight, free from proteins and naturally-occurring organic molecules with which it is naturally associated. Preferably, the preparation is at least 75%, more preferably 90%, and most preferably at least 99%, by weight, antibody. A purified antibody of the invention may be obtained, for example, by affinity chromatography using a recombinantly-produced polypeptide of the invention and standard techniques.

By “specifically binds” is meant a compound or antibody which recognizes and binds a polypeptide of the invention but which does not substantially recognize and bind other molecules in a sample, for example, a biological sample, which naturally includes a polypeptide of the invention.

By “derived from” is meant isolated from or having the sequence of a naturally-occurring sequence (e.g., a cDNA, genomic DNA, synthetic, or combination thereof).

By “inhibiting a pathogen” is meant the ability of a candidate compound to decrease, suppress, attenuate, diminish, or arrest the development or progression of a pathogen-mediated disease or infection in a eukaryotic host organism. Preferably, such inhibition decreases pathogenicity by at least 5%, more preferably by at least 25%, and most preferably by at least 50%, as compared to symptoms in the absence of the candidate compound in any appropriate pathogenicity assay (for example, those assays described herein). In one particular example, inhibition may be measured by monitoring pathogenic symptoms in a host organism exposed to a candidate compound or extract, a decrease in the level of symptoms relative to the level of pathogenic symptoms in a host organism not exposed to the compound indicating compound-mediated inhibition of the pathogen.

By “pathogenic virulence factor” is meant a cellular component (e.g., a protein such as a transcription factor, as well as the gene which encodes such a protein) without which the pathogen is incapable of causing disease or infection in a eukaryotic host organism.

The invention provides a number of targets that are useful for the development of drugs that specifically block the pathogenicity of a microbe. In addition, the methods of the invention provide a facile means to identify compounds that are safe for use in eukaryotic host organisms (i.e., compounds which do not adversely affect the normal development and physiology of the organism), and efficacious against pathogenic microbes (i.e., by suppressing the virulence of a pathogen). In addition, the methods of the invention provide a route for analyzing virtually any number of compounds for an anti-virulence effect with high-volume throughput, high sensitivity, and low complexity. The methods are also relatively inexpensive to perform and enable the analysis of small quantities of active substances found in either purified or crude extract form.

Other features and advantages of the invention will be apparent from the detailed description, and from the claims.

### BRIEF DESCRIPTION OF THE DRAWINGS

FIG. 1 shows the separation of representational difference analysis, “RDA,” products by agarose gel electrophoresis between two strains of P. aeruginosa, PA14 and PA01, by the tester DNA amplicons before DNA hybridization and amplification (a) and the difference products after the first (b) and second (c) DNA hybridization-PCR amplification steps. Molecular weight (M) marker sizes are indicated on the left in base pairs.

FIG. 2 shows a Southern blot analysis confirming that the second round RDA products are unique to PA14. Chromosomal DNA was isolated from P. aeruginosa strains PA01 and PA14, digested with Sau3A1, separated on a 0.8% agarose gel, transferred to a membrane, and hybridized with a labeled pool of the second-round products. Molecular weight markers are indicated on the left in kilobase pairs.

FIG. 3 shows the physical map and nucleotide sequence of the ybtQ gene (SEQ ID NO: 1) from P. aeruginosa strain PA14. The deduced protein sequence (SEQ ID NO:2) is shown below the coding region of ybtQ. The numbering refers to the letter of the nucleotide sequence. The 196-bp of the RDA product recovered in pJY11 is underlined. The asterisks bracket the ABC transporter domain and the four boxed regions show the conserved motifs of the ABC transporter domain (Walker A, ABC signature, Walker B, and an unnamed fourth motif), as defined by Linton and Higgins (Higgins, C. F. Annu. Rev. Cell Biol. 8:67-113, 1992; Linton et al., Mol. Microbiol. 28:5-13, 1998).

FIG. 4 shows an alignment of the YbtQ homolog of P. aeruginosa PA14 (upper line) with YbtQ of Y. pestis (YP; SEQ ID NO: 35). This alignment was generated with Clustal W. Dark boxes indicate identical residues and light boxes enclose similar residues. Numbers above each pair of sequences reflect the deduced protein sequence of the YbtQ homolog in P. aeruginosa PA14.

FIG. 5A depicts the LD₅₀s of P. aeruginosa strains PA14 and PA01 and four mutants in fifth-instar G. mellonella larvae. Ten larvae were injected at each dilution (containing 0-10⁶ bacteria) and larvae were scored as live or dead after sixty hours at 25° C. The data represent the means and the standard deviations of three independent experiments. Asterisks indicate statistically significant differences between the mutant strains and P. aeruginosa strain PA 14.

FIG. 5B shows the mortality (%) in a burned mouse model for P. aeruginosa strains PA14 and PA01, and for four mutants. Eight mice per experiment were injected subcutaneously with 5×10⁵ CFU of each P. aeruginosa strain and the number of animals that died as a result of sepsis was monitored each day for ten days. The data represent the means and standard deviations of two independent experiments for PA14 and the four mutants; PA01 was tested once. Asterisks indicate statistically significant differences of mutants from PA14.

FIG. 6 shows the pilA nucleic acid (SEQ ID NO:3) and amino acid (SEQ ID NO:4) sequence. The deduced protein sequence is shown below the coding region of pilA.

FIG. 7 shows the pilC nucleic acid (SEQ ID NO:5) and amino acid (SEQ ID NO:6) sequence. The deduced protein sequence is shown below the coding region of pilC.

FIG. 8 shows the uvrD (SEQ ID NO:7) nucleic acid and amino acid (SEQ ID NO:8) sequence. The deduced protein sequence is shown below the coding region of uvrD.

### DETAILED DESCRIPTION

To identify virulence genes in bacteria the natural differences in pathogenicity between isolates of the same species may be used. The genomes of the strains may be compared using a subtractive hybridization technique to recover relevant genomic differences. The sequenced strain of P. aeruginosa, strain PA01, has substantial differences in virulence from strain PA14. As is described in more detail below, the technique of RDA was used to recover genomic differences between P. aeruginosa strains PA14 and PA01. The pilC, pilA, and uvrD genes in PA14 differed from their counterparts in strain PA01. In addition, a gene homologous to the ybtQ gene from Yersinia pestis was recovered, and mutation of this ybtQ homolog was found to attenuate the virulence of the PA14 strain. These experimental examples are intended to illustrate, not limit, the scope of the claimed invention.

Identification of PA14-Specific Nucleic Acid Fragments

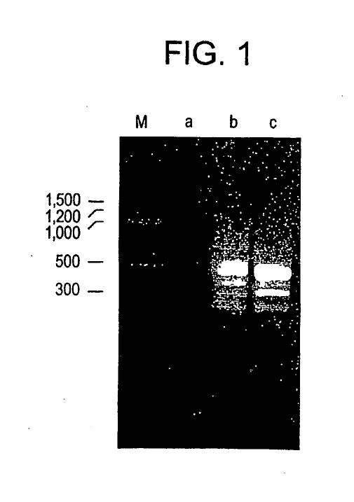

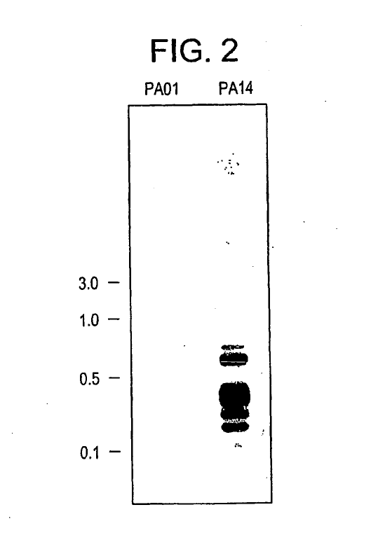

A library of fragments of P. aeruginosa PA14 DNA was constructed according to standard methods. Using the technique of RDA total genomic DNA of the sequenced strain, P. aeruginosa PA01, was subtracted from strain PA14. A Southern blot was used to confirm that the sequences amplified by RDA were specific to P. aeruginosa PA14. FIG. 1 shows the separation of RDA products by agarose gel electrophoresis between the two strains of P. aeruginosa, PA14 and PA01, by the tester DNA amplicons before DNA hybridization and amplification (a) and the difference products after the first (b) and second (c) DNA hybridization-PCR amplification steps. FIG. 2 shows a Southern blot analysis confirming that the second round RDA products are unique to PA14. These RDA amplicons were then ligated into pEX18, and twenty clones, were sequenced using primers flanking the polylinker site of pEX18. When the sequences of these twenty inserts were compared to the P. aeruginosa PA01 genome sequence (Genbank accession number: NC_{—}002516) (http://www.pseudomonas.com/GenomeSearch.asp) none showed a match, as expected from the Southern results. Of the twenty clones chosen for further analysis, fourteen showed homology to known genes, while five had no significant matches by BLAST search (http://www.ncbi.nlm.nih.gov/blast) (Table 1).

TABLE 1
Homologies of the P. aeruginosa PA14
strain-specific RDA clones

| Clone | | | GenBank |
|---|---|---|---|
| No. | Length (bp) | Homologies with BLASTX | Accession No. |
| pJY1 | 195-bp | Ubiquitin-specific protease; Ubp7p | NC001141 |
| | | of Saccharomyces cerevisiae | |
| pJY2 | 175-bp | PilC of Pseudomonas aeruginosa | M32066 |
| pJY3 | 138-bp | Cholorophyll b synthetase of Dunaliella salina | AB021312 |
| pJY4 | 338-bp | KIAA0054 gene product; helicase | XP008261 |
| | | of Homo sapiens | |
| pJY5 | 348-bp | Type 4 pilin of Pseudomonas aeruginosa | L37109 |
| pJY6 | 180-bp | Unknown | |
| PJY8 | 397-bp | Hypothetical protein Y68A4A.10 | AL021503 |
| | | of Caenorhabditis elegans | |
| pJY10 | 117-bp | Unknown | |
| pJY11 | 196-bp | YbtQ, ABC transporter, ATP-binding component | AF091251 |
| | | of Yersinia pestis | |
| pJY12 | 234-bp | Unknown | |
| pJY13 | 215-bp | Alpha-1 tubulin of Caenorhabditis elegans | D16439 |
| pJY15 | 368-bp | UvrD, DNA helicase of Chlamydia trachomatis | AE001331 |
| pJY16 | 116-bp | Unknown | |
| pJY17 | 230-bp | Patched-related proteins of Caenorhabditis elegans | AC006670 |
| pJY19 | 274-bp | Unknown protein of Pasteurella multocida | AE006141 |
| pJY20 | 128-bp | PilC of Neisseria gonorrhoeae | AJ00121 |
| pJY22 | 217-bp | Unknown | |
| pJY23 | 264-bp | Nuclear receptor NHR-18 of Caenorhabditis elegans | AF083232 |
| pJY24 | 366-bp | Hypothetical 119.5 K protein of Micrococcus luteus | JQ0405 |
| pJY25 | 215-bp | Alpha-1 tubulin of Caenorhabditis elegans | D16439 |

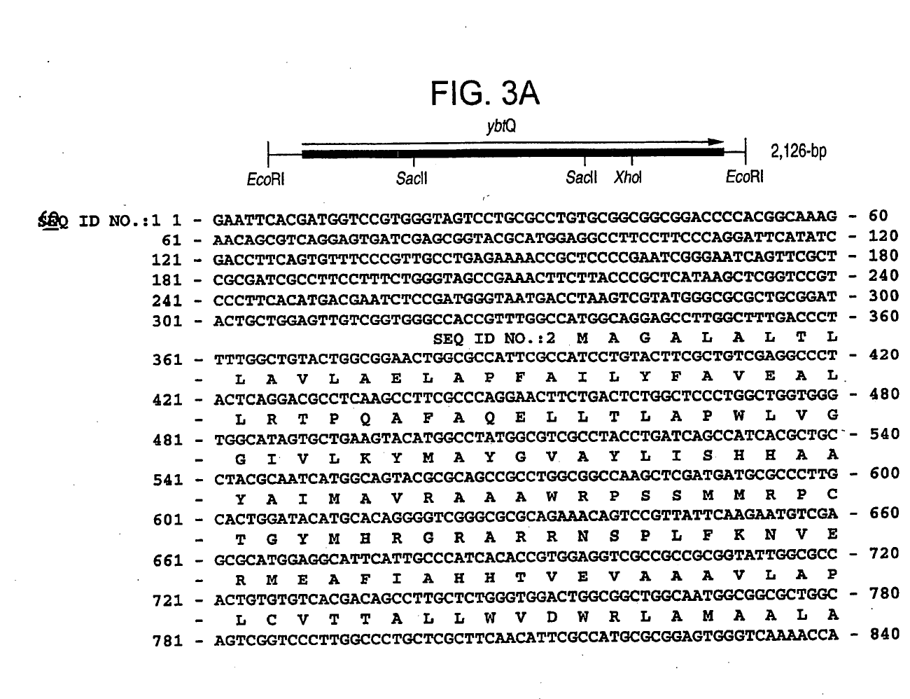

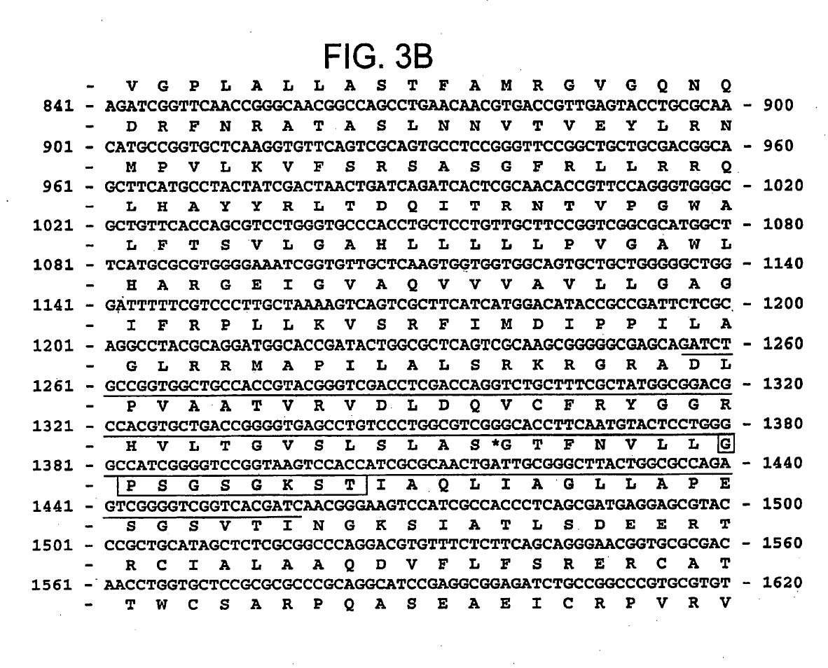

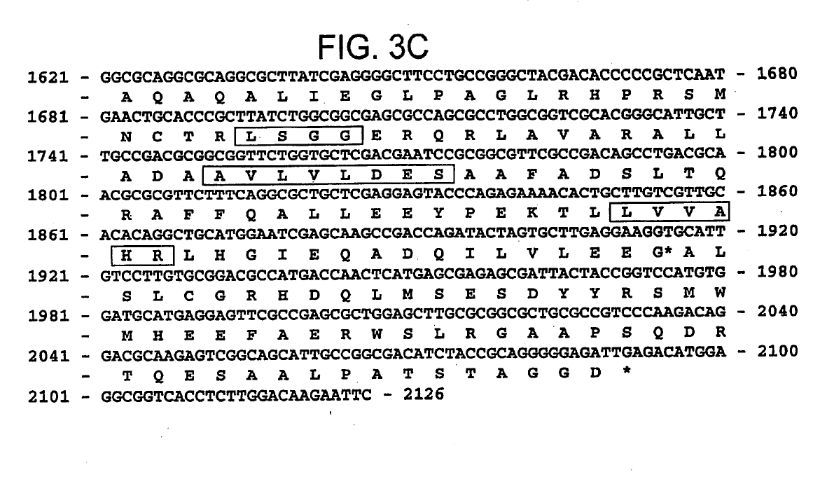

A gene homologous to the ybtQ gene from Yersinia was recovered that is specifically present in strain PA14, but absent in strain PA01. FIG. 3 shows the physical map and nucleotide sequence of ybtQ gene (SEQ ID NO: 1) The deduced protein sequence is shown below the coding region of ybtQ. Mutation of the ybtQ homolog in P. aeruginosa strain PA14 significantly attenuates the virulence of this strain in both G. mellonella and a burned mouse model of sepsis, to levels comparable to those seen with PA01. This suggests that the increased virulence of P. aeruginosa strain PA14, as compared to PA01, may relate to specific genomic differences identified by RDA.

The pilC, pilA, and uvrD genes in strain PA14 were found to differ substantially from their counterparts in strain PA01. Attention was then focused on the inserts in pJY2, pJY5, pJY11, and pJY15 that BLAST searches revealed to be homologs respectively of the type IV fimbrial assembly protein, PilC, in P. aeruginosa; the type IV pilin, PilA, in P. aeruginosa; the ABC-transporter protein, YbtQ, in Yersinia pestis, and the DNA helicase, UvrD, in Chlamydia trachomatis.

Cloning and Sequencing of the pilA, pilC, and uvrD Genes from P. Aeruginosa Strain PA14

1,030-bp (SEQ ID NO:3), 1,160-bp (SEQ ID NO: 5), and 1,150-bp (SEQ ID NO:7) fragments containing P. aeruginosa strain PA14 pilA, pilC, and uvrD genes were recovered by inverse PCR, and cloned into pGEM-T Easy to construct plasmids pJY2A, pJY5A, and pJY15A, respectively.

Using standard blast analysis, the deduced amino acid sequence (SEQ ID NO:6) encoded by the 1,160 bp insert of pJY2A, showed 72% identity and 79% similarity to the type IV fimbrial assembly protein PilC in P. aeruginosa strain PA01. A pair wise BLAST alignment (http://www.ncbi.nlm.nih.gov/blast/bl2seq/bl2.html) of the PilC amino acid sequences from P. aeruginosa strains PA01 and PA14 showed these genes shared a number of variable regions separated by conserved domains; the RDA product originally isolated in pJY2 was derived from one of the variable regions that differs substantially between PA14 and PA01.

A BLAST search using the insert in pJY5A (SEQ ID NO:4) showed 91% identity over 179 amino acids to the type IV pilin from the P. aeruginosa G7 and G9 strains (Spangenberg, et al., FEMS Microbiol. Lett. 125:265-274, 1995). The N terminal 30 amino acids showed significant homology to various other members of the type IV group A prepilins, including PilA of P. aeruginosa strain PA01 (Johnson et al., J. Biol. Chem. 261:15703-15708, 1986), FimA of Dichelobacter nodosus (Billington et al., Gene 99:115-119, 1991), and PilE of N. gonorrhoeae (Bergstrom et al., Proc. Natl. Acad. Sci. U.S.A 83:3890-3894, 1986). The C-terminal regions of these proteins were more variable, and the RDA product originally isolated in pJY5 was from a region quite variable in the pilA genes of PA14 and PA01. Overall, the genes pilA from PA14 and PA01 were only 53% similar to each other.

A 3.0-kbp fragment of the pilABC gene cluster was recovered from PA14 in plasmid pJYP25, and sequence analysis demonstrated that the pilC gene in pJY2A and the pilA gene in pJY5A were linked in a pilABC gene cluster that was otherwise nearly identical to the same cluster in PA01.

A BLAST search of the insert in pJY15A (SEQ ID NO:7) showed high levels of similarity to the DNA helicase (UvrD) of Chlamydia trachomatis (27% identity and 45% similarity over 902-bp), the DNA helicase of Borrelia burgdorferi (22% identity and 42% similarity over 758-bp) as well as other DNA helicase family members from other organisms. P. aeruginosa strain PA01 also has uvrD in its genome (http://www.pseudomonas.com/GenomeSearch.asp), but there is only 45% similarity between the individual uvrD genes found in P. aeruginosa strains PA14 and PA01. The insert from pJY15A came from an area that was particularly divergent between the sequences of the single uvrD genes in these two strains.

Analysis of the ybtQ Gene Homolog in P. Aeruginosa strain PA14

DNA sequence analysis of pJY11 showed that the insert was 196 nucleotides in length and had homology to the ybtQ gene of Y. pestis. This insert was used to recover a 2.1 kilobase pair fragment of P. aeruginosa strain PA14 chromosomal DNA overlapping the insert in pJY11 that was cloned into plasmid pJYYBT. Sequencing of pJYYBT revealed a 2,126-bp complete open reading frame (SEQ ID NO: 1) encoding a protein of 585 amino acids (SEQ ID NO:2) with a predicted molecular mass of 63,549 Da, and a pI of 9.57 (FIG. 3). A BLAST search revealed that the highest homologies of this open reading frame was most homologous with the ABC-transporter protein, YbtQ, of Y. pestis (24% identity and 38% similarity in a 1,061-bp overlap); the inner membrane ABC-transporter, Irp7, of Y. enterocolitica (24% identity and 38% similarity over 962-bp); the ABC-transporter protein, YbtP, of Y. pestis (21% identity and 36% similarity over 1,061-bp); and the ABC-transporter protein, Irp6, of Y. enterocolitica (21% identify and 36% similarity over 1,061 bp). The genes encoding the Ybt systems of Y. pestis and Y. enterocolitica showed greater than 97% sequence identity (Buchrieser et al., Infect Immun. 67:4851-4861, 1999; Gehring et al., Chem. Biol. 5:573-586, 1998; Gehring et al., Biochemistry 37:11637-11650, 1998; Rakin et al., Infect. Immun. 67:5265-5274, 1999; Schubert et al., Infect. Immun. 66:480-485, 1998), and ybtP and ybtQ in Y. pestis are orthologs of irp6 and irp7 in Y. enterocolitica. Similarity analyses of deduced amino acid sequences by the WU-Blast2 program in EMBL (European Bioinformatics Institute) (http://www2.ebi.ac.uk/blast2/) also revealed that the amino acid sequence of the protein encoded in pJYYBT showed 30.7%, 31.2%, 28.6%, and 28.8% similarity to YbtQ, Irp7, YbtP, and Irp6 respectively.

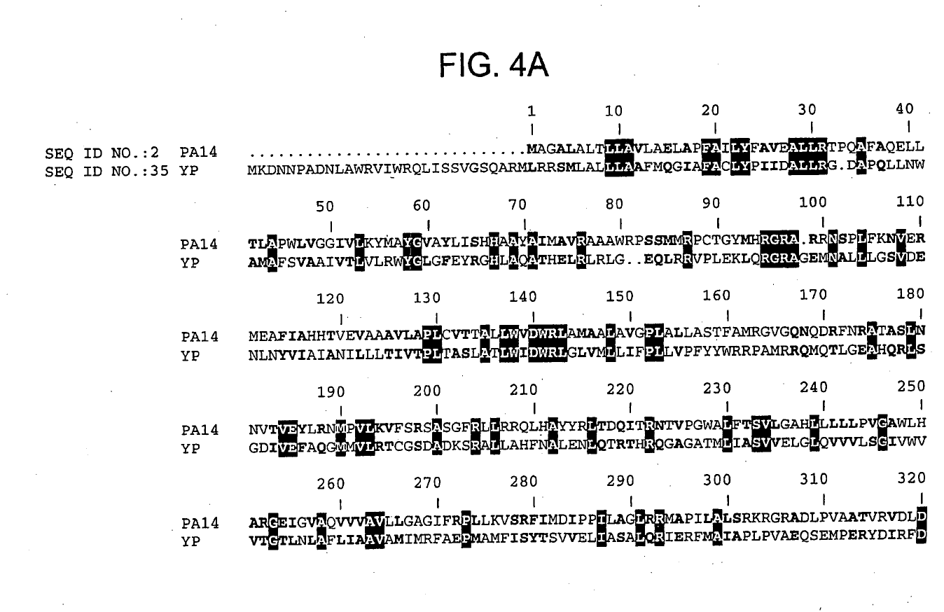

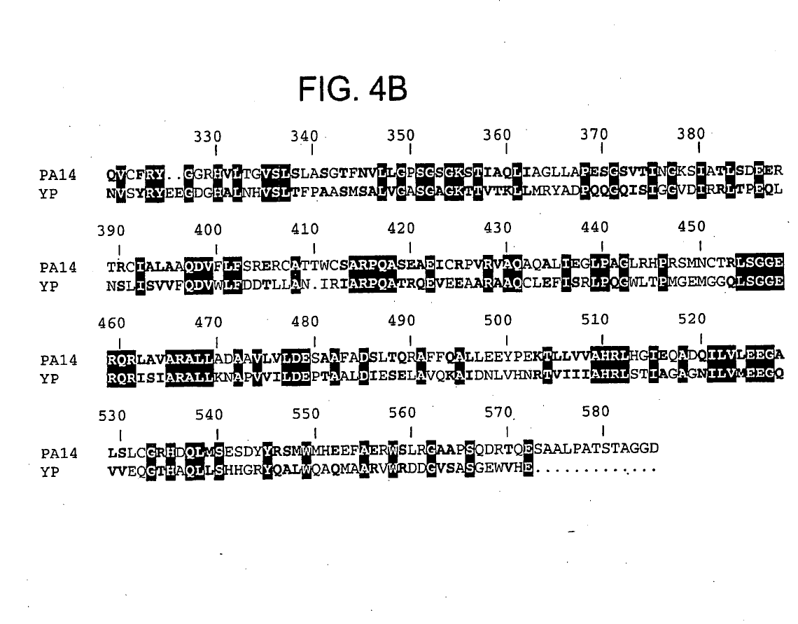

The N-terminal region of the YbtQ protein in P. aeruginosa PA14 was not very well conserved with respect to that of YbtQ in Y. pestis. While the C-terminal region, which is hypothesized to act as a signal sensor (FIG. 4), showed high similarity. Analysis of the deduced amino acid sequence of the open reading frame in pJYYBT by the Expert Protein Analysis System (http://www.expasy.ch) suggested an amino-terminal hydrophobic region with six possible transmembrane segments, as well as an ABC transporter signature motif in the carboxy-terminal portion of the protein (FIG. 3) (Ames et al., Adv. Enzymol. Relat. Areas Mol. Biol. 65:1-47, 1992; Higgins, C. F. Annu. Rev. Cell Biol. 8:67-113, 1992; Linton et al., Mol. Microbiol. 28:5-13, 1998).

Nucleotide sequence comparison with the genome sequence database of PA01 (http://www.pseudomonas.com/GenomeSearch.asp) revealed no sequences corresponding to the ybtQ homolog in PA14, suggesting that the entire ybtQ gene (and possibly surrounding sequences) was uniquely present in P. aeruginosa strain PA14 but absent from PA01. The ybtQ gene sequence from PA14 was compared with the sequence of a recently described pathogenicity island, PAGI, present in the majority of pathogenic isolates of P. aeruginosa (Liang et al., J. Bacteriol. 183:843-853, 2001), but no sequences homologous to ybtQ in PAGI were found.

Construction of Mutants in the pilC, pilA, uvrD, and ybtQ Genes of PA14 and Determination of Phenotypes

Standard methods were used to insertionally inactivate the pilC, pilA, uvrD, and ybtQ genes of P. aeruginosa strain PA14. Plasmids pJY2M, pJY5M, pJY11M, and pJY15M, encoding 5′- and 3′-truncated pilC, pilA, uvrD, and ybtQ genes, respectively, were integrated into the genome of P. aeruginosa strain PA14 by single homologous recombination events. The insertional disruption of the appropriate gene in the corresponding mutant was confirmed by Southern blot analysis.

Virulence Testing of the Four Mutants in both Non-Vertebrate and Mouse Models of Infection

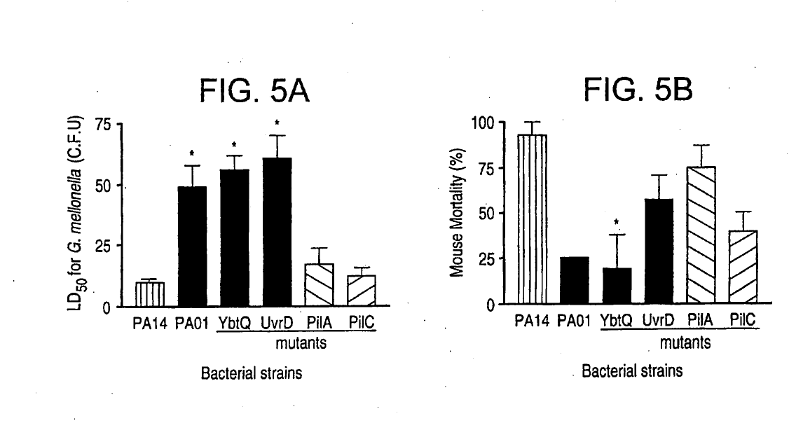

The strains PA14 and PA01 show differences in a number of models of virulence, including the slow killing of C. elegans, killing of wax moth caterpillars, and in a burned mouse model of sepsis. It was useful to determine if the differences in virulence of these two P. aeruginosa strains in these various models related to the genetic differences uncovered by RDA. The virulence of PA14, PA01, and PA14 mutants in pilC, pilA, uvrD, and ybtQ was then assayed. In the C. elegans slow killing model, there were no differences in virulence between strain PA14 and the mutants JY2M, JY5M, JY11M, and JY15M, respectively. However, when the LD₅₀s of P. aeruginosa strains PA14, PA01, and the four mutants in G. mellonella (the wax caterpillar), the uvrD and ybtQ mutants of PA14 were determined, they exhibited attenuated virulence, very similar to that of PA01 (FIG. 5A). The LD₅₀ for G. mellonella for these three strains were all approximately one log higher than for PA14 or the mutants in pilA or pilC (p<0.05).

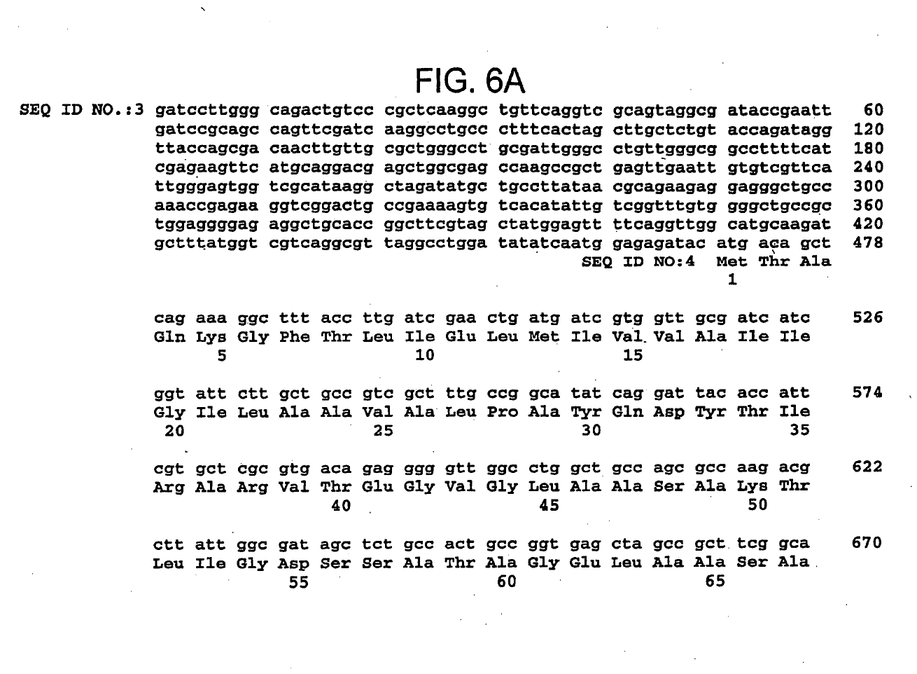

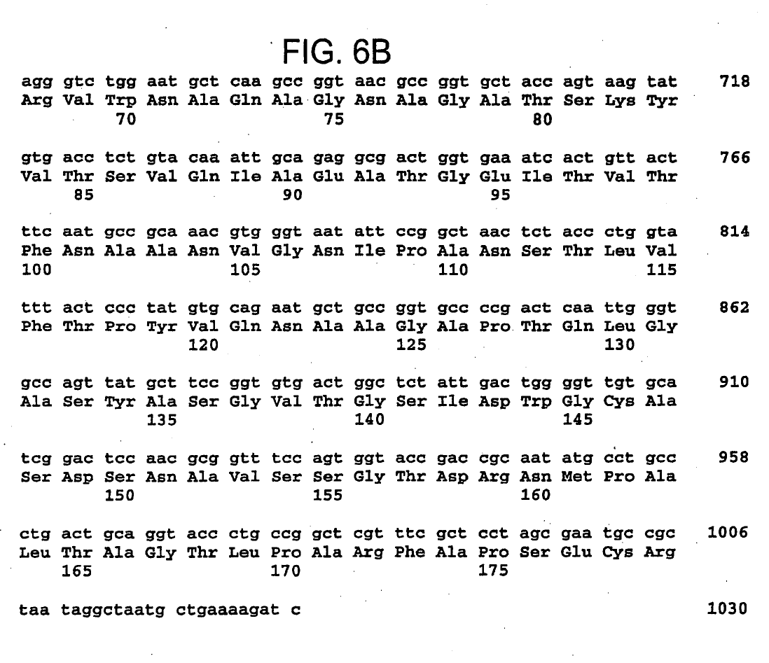

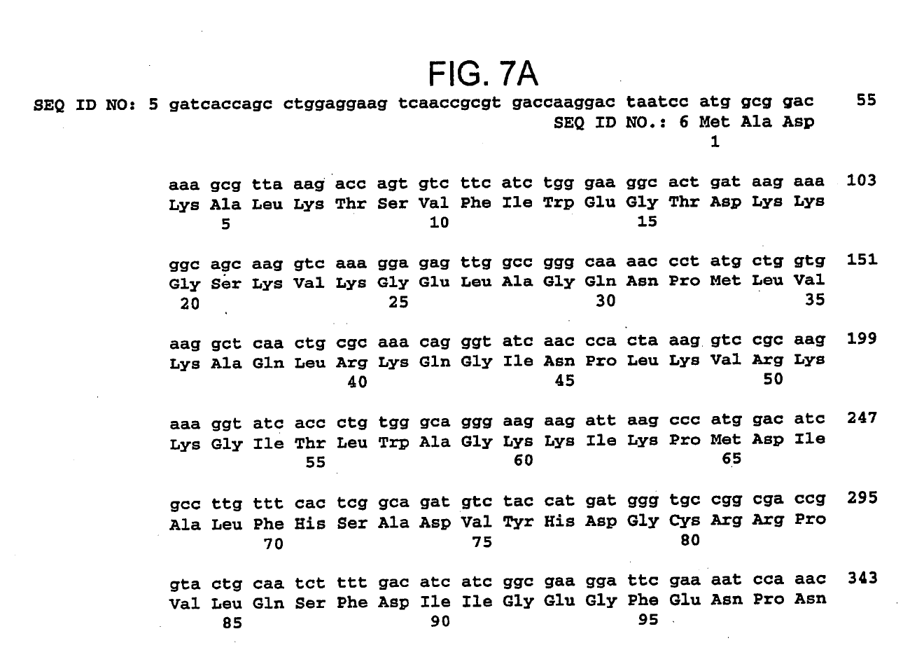

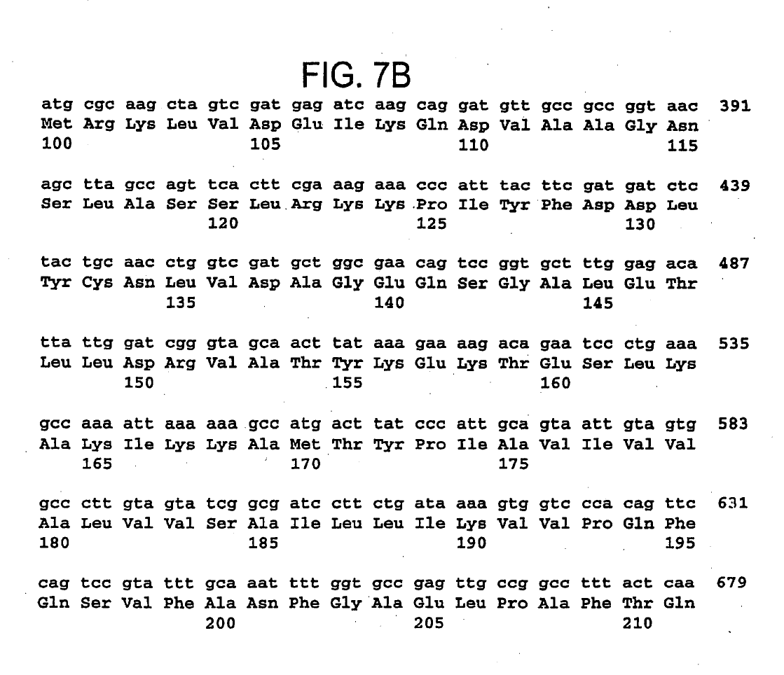

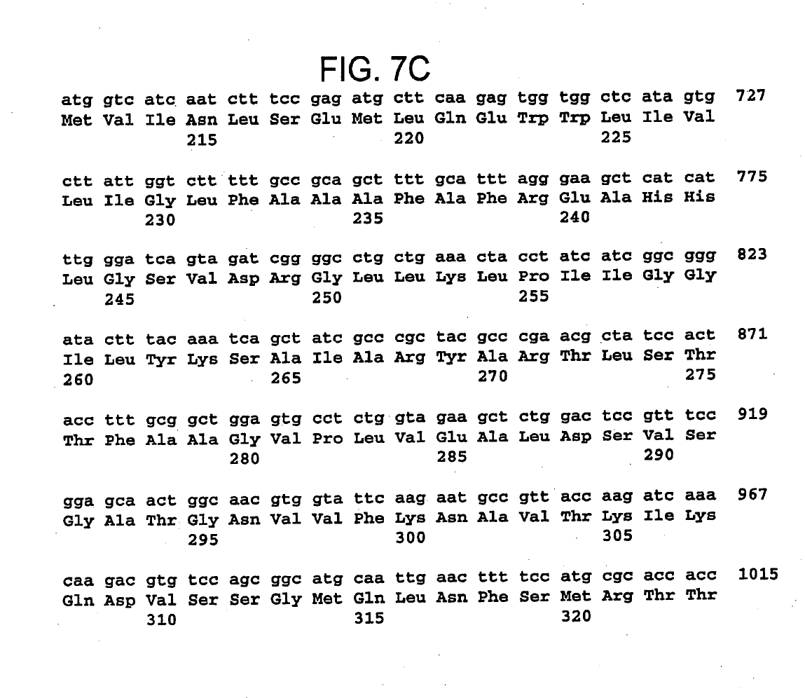

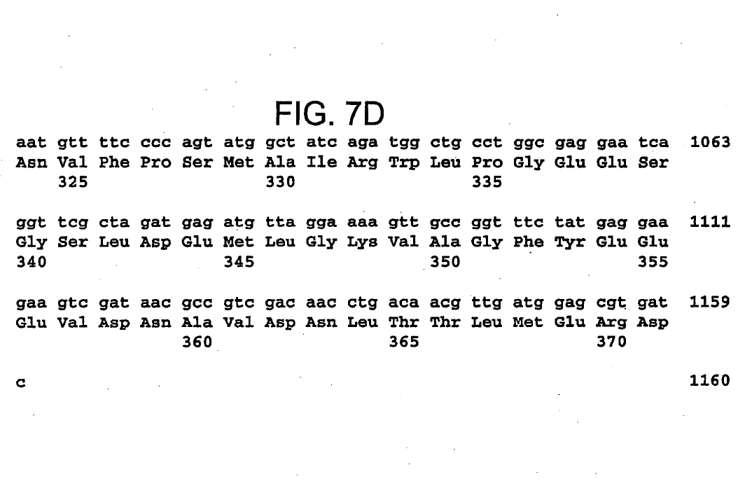

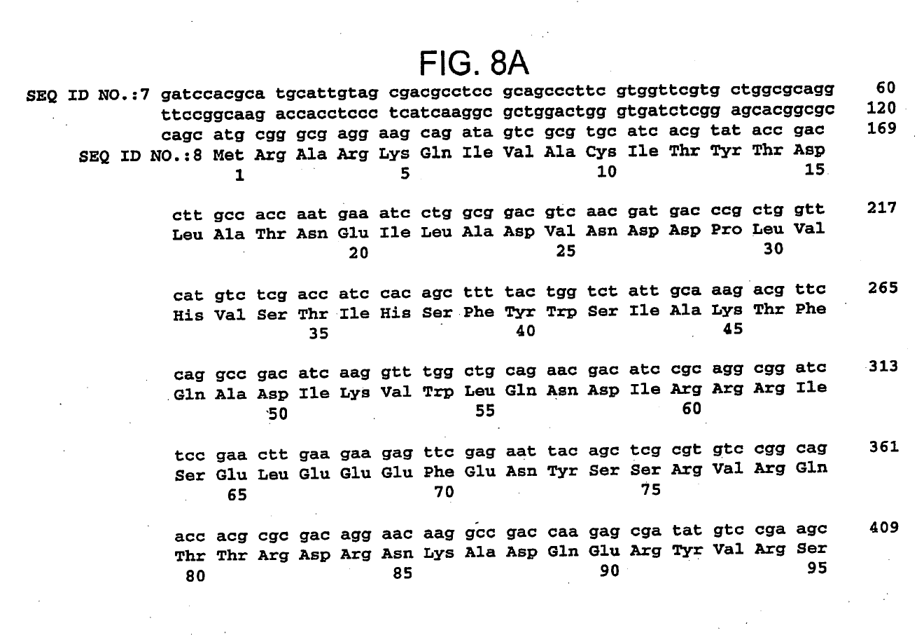

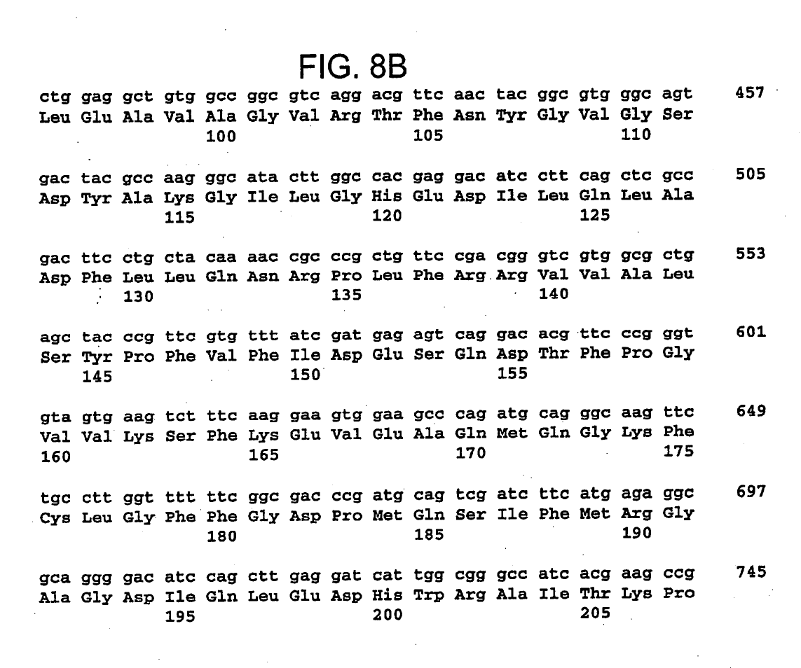

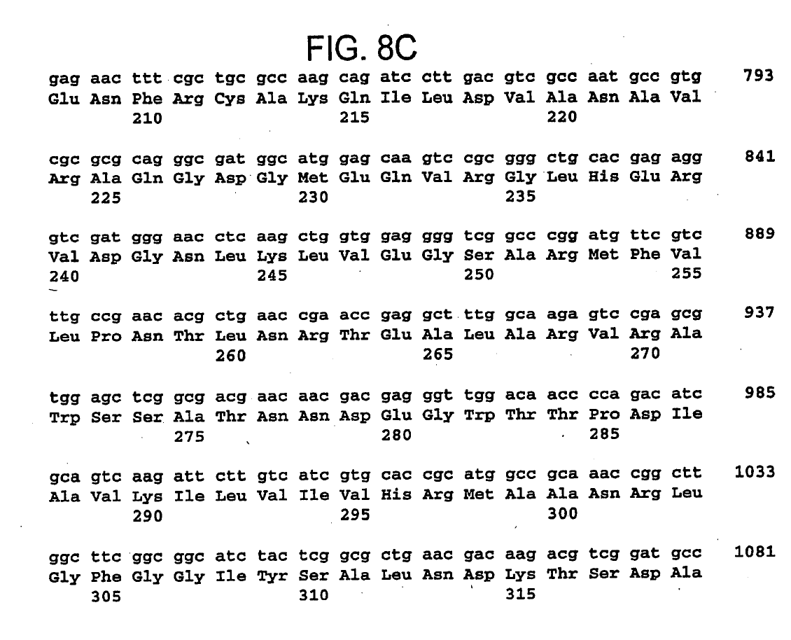

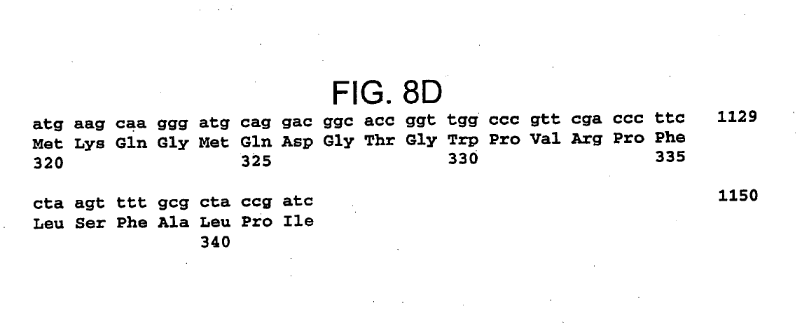

Rahme et al. (Science 268:1899-1902, 1995), have previously reported that P. aeruginosa strain PA14 is more virulent in a burned mouse model of sepsis than PA01, and this observation was confirmed (FIG. 5B). The nucleic acid and deduced amino acid sequences of pilA, pilC, and uvrD are shown in FIGS. 6, 7, and 8, respectively. When the virulence of the pilA, pilC, uvrD and ybtQ mutants of PA14 in this model was examined, the ybtQ mutant was significantly attenuated, comparable to the attenuation seen with PA01. Thus, a mutation of ybtQ in P. aeruginosa strain PA14, a gene missing in strain PA01 (and identified by RDA), significantly attenuated the virulence of strain PA14 in two different model systems of infection, to levels comparable to that seen with PA01.

In Yersinia, YbtP and YbtQ are involved in iron acquisition from the environment. The ability of the ybtQ mutant of PA14 (strain JY11), to grow in iron-chelated syncase media was then examined. However, no differences were detected in growth in this media compared to wild-type strain PA14, perhaps reflecting the many other iron acquisition systems present in P. aeruginosa.

### SUMMARY

The hypothesis that differences in pathogenesis between specific strains of P. aeruginosa may reflect discrete genomic differences identifiable by RDA was examined. Recently, RDA has been used to detect differences between virulent and avirulent Mycobacterium bovis strains (Mahairas et al., J. Bacteriol. 178:1274-1282, 1996) and between Neisseria gonorrhoeae and N. meningitidis (Tinsley et al., Proc. Natl. Acad. Sci. U.S.A. 93:11109-11114, 1996). Previous studies have demonstrated that P. aeruginosa strain PA14 is significantly more virulent in a slow killing assay with C. elegans (Tan et al., Proc. Natl. Acad. Sci. U.S.A. 96:715-720, 1999) and in a burned mouse model of infection (Rahme et al., Science 268:1899-1902, 1995), as compared to the well-characterized and sequenced strain, PA01. These differences in pathogenesis were utilized, in combination with RDA, to examine specific genes in strain PA14 that might underlie differences in pathogenesis from strain PA01. Differences in the pilC, pilA, and uvrD genes were identified between P. aeruginosa strains PA14 and PA01. Also a ybtQ homolog in strain PA14 was identified that was entirely missing in strain PA01. Mutations were constructed in each of these four genes, and the pathogenesis of these mutants was compared with that of the parent strain, PA14, and the reference strain, PA01, in both non-vertebrate model systems and a mouse model of burn infection.

Mutations of the pilC and pilA genes in strain PA14 did not alter the virulence of the strains in the various model systems tested. The pilC and pilA genes in P. aeruginosa strain PA 14 were located in the same pilABC cluster as in strain PA01, but were divergent at the sequence level; additional homologs of pilA and pilC were not identified in P aeruginosa strain PA14 by Southern blot (data not shown). When the deduced amino acid sequence of PilC from P. aeruginosa strain PA14 was compared with that from strain PA01, the proteins were shown to have very high homology in a number of conserved domains, separated by a number of variable regions. This structure is reminiscent of the PilE protein in N. gonorrhoeae, which has been shown to undergo pilus antigenic variation. This antigenic variation occurs by the high-frequency, unidirectional transfer of DNA sequences from one of several silent pilin loci (pilS genes) into the expressed pilin gene locus (pilE), resulting in changes in the primary pilin protein sequence (Bergstrom et al., Proc. Natl. Acad. Sci. U.S.A. 83:3890-3894, 1986; Meyer et al., Annu. Rev. Microbiol. 44: 451477, 1990; Swanson et al., J. Exp. Med. 162:729-744, 1985; Swanson et al., Cell 47:267-276, 1986). Since no additional pilC homologs in P. aeruginosa were detected by Southern blot, the mechanism of divergence between the pilC genes in P. aeruginosa strains PA14 and PA01 is, currently, uncertain.

The PA14 uvrD mutant was attenuated in virulence in G. mellonella, but not in C. elegans or the burned mouse model, whereas the ybtQ mutant was attenuated for virulence in both G. mellonella and in the burned mouse model (but not in C. elegans). Previous studies of P. aeruginosa mutants that were tested in both C. elegans and in G. mellonella have shown that several mutants exhibit attenuated pathogenesis specifically in one host, but not in the other (Jander et al., J. Bacteriol. 182: 3843-3845, 2000; Tan et al., Proc. Natl. Acad. Sci. U.S.A. 96:715-720, 1999; Tan et al., Proc. Natl. Acad. Sci. U.S.A. 96:2408-2413, 1999). This suggests that screening for pathogenesis in a variety of hosts may lead to the identification of subsets of virulence factors that are critical for infection in specific hosts, as well as others that are important for infection in all hosts. For example, Mahajan-Miklos et al. (Mol. Microbiol. 37: 981-988, 2000) have suggested that screening for mutants of P. aeruginosa attenuated in a fast-killing assay of C. elegans under hyperosmolar and low pH conditions may identify genes that are also important for virulence in a cystic fibrosis lung infection model.

P. aeruginosa utilizes several siderophores for high-affinity iron uptake, including pyoverdin and pyochelin (Cox et al., Infect. Immun. 48:130-138, 1985; Cox et al., J. Bacteriol. 137:357-364, 1979; Poole et al., Molecular Biology of Pseudomonas, pages 371-383, Washington D.C., American Society for Microbiology, 1996). Pyoverdin production has been previously shown to be required for bacterial colonization of the lung in a rat infection model (Poole et al., Molecular biology of Pseudomonas, pages 371-383, Washington D.C., American Society for Microbiology, 1996) and to correlate with lethality in a burned mouse infection model (Meyer et al., Infect. Immun. 64:518-523, 1996). Pyochelin has been shown to promote bacterial growth and lethality when injected into the peritoneal cavities of mice simultaneously with an avirulent mutant of P. aeruginosa strain PA01, isolated by repetitive passage in mice (Cox, C.D. Infect. Immun. 36:17-23, 1982).

Yersiniabactin, a phenolate-thiazole siderophore, was first purified from Yersinia enterocolitica and subsequently from Y. pestis. In Y. pestis, yersiniabactin may play a role in establishing infection at the site of a flea bite, as mutants that are unable to produce or transport yersiniabactin are avirulent in mice by peripheral routes of infection, but fully virulent when infected intravenously (Bearden et al., Infect. Immun. 65:1659-1668, 1997). Several of the genes needed for the biosynthesis and utilization of yersiniabactin are clustered in a pathogenicity island that is part of an unstable region on the Y. pestis chromosome, the pgm locus (Bearden et al., Infect. Immun. 65:1659-1668, 1997; Fetherston et al., Mol. Microbiol. 32:289-299, 1999; Gehring et al., Chem. Biol. 5:573-586, 1998; Gehring et al., Biochemistry 37:11637-11650, 1998; Pelludat et al. J. Bacteriol. 180:538-546, 1998). All highly pathogenic species of Yersinia have similar genes clustered on a high-pathogenicity island (HPI). P. aeruginosa has not previously been shown to have genes homologous to the yersiniabactin system of Yersinia.

Our results suggests that the ybtQ homolog that is present in strain PA14, but absent in strain PA01 may at least partially explain the differences in pathogenesis of these two strains for the burned mouse model as well as for G. mellonella. P. aeruginosa strain PA14 may contain a pathogenicity island encoding ybtQ and other genes that is absent from strain PA01 and was acquired by horizontal gene transfer. Extensive genomic rearrangements, as well as acquisition and loss of large blocks of DNA, have previously been demonstrated in different P. aeruginosa isolates (Romling et al., J. Mol. Biol. 271:386-404, 1997).

Materials and Methods

Described below are detailed materials and methods relating to the above-described identification of Pseudomonas aeruginosa PA14 virulence genes, and the testing of these genes for virulence in nematode life span and mouse burn assays.

Bacterial Strains

Bacterial strains and plasmids used in this study are shown in Table 2. P. aeruginosa strain PA14 is a human clinical isolate used for identification of novel virulence-related genes. P. aeruginosa strain PA01 has been studied extensively in many laboratories (Hassett D. J., J. Bacteriol. 178:7322-7325, 1996; Nicas et al., Can. J Microbiol. 31:387-392, 1985; Ohman et al., J. Infect. Dis. 142:547-555,1980; Ostroff et al., Infect. Immun. 57:1369-1373, 1989) and the genomic sequence has been determined (Stover et al., Nature 406: 959-964, 2000). All strains were maintained at −70° C. in Luria-Bertani (LB) medium containing 15% glycerol. LB broth and agar were used for the growth of P. aeruginosa and Escherichia coli strains at 37° C. Chelex-100 treated syncase media (Finkelstein et al., J. Immunol. 96:440-449, 1966) was used for the low iron growth conditions for the P. aeruginosa ybtQ mutant. Antibiotic concentrations were as follows: for E. coli, ampicillin (100 μg ml⁻¹); for P. aeruginosa, rifampicin (100 μg ml⁻¹) and carbenicillin (300 μg ml⁻¹).

TABLE 2
Bacterial strains and plasmids used in this study

| Strain or plasmid | Relevant genotype and/or phenotype | Source or reference |
|---|---|---|
| P. aeruginosa | | |
| PA01 | Wild-type laboratory strain | Lab collection |
| PA14 | Human clinical isolate; Rif^{r} | 45 |
| JY2M | pilC mutant of PA14; Rif^{r} Cb^{r} | This study |
| JY5M | pilA mutant of PA14; Rif^{r} Cb^{r} | This study |
| JY11M | ybtQ mutant of PA14; Rif^{r} Cb^{r} | This study |
| JY15M | uvrD mutant of PA14; Rif^{r} Cb^{r} | This study |
| E. coli | | |
| DH5 α | F endA1 hsdR17 supE44 thi-1 recA1 | Gibco BRL |
| | gyrA96 relA1Δ(lacZYA-argF) U-169 | |
| | λ-o80 dlacZΔM15 | |
| SM10 | thi-1 thi leu tonA lacY supE recA:: | Lab collection |
| | RP4-2-Tc::Mu; Km^{r} | |
| Plasmids | | |
| pUC19 | Cloning vector; Ap^{r} | Lab collection |
| pEX18 | Suicide vector for P. aeruginosa; oriT⁺ sacB⁺, | 23 |
| | gene replacement vector with MCS from pUC18; Ap^{r} | |
| pGEM-T Easy | PCR cloning vector; Ap^{r} | Promega |
| pJY1 | pEX18 derivative carrying RDA product; Ap^{r} | This study |
| pJY2 | pEX18 derivative carrying RDA product; Ap^{r} | This study |
| pJY3 | pEX18 derivative carrying RDA product; Ap^{r} | This study |
| pJY4 | pEX18 derivative carrying RDA product; Ap^{r} | This study |
| pJY5 | pEX18 derivative carrying RDA product; Ap^{r} | This study |
| pJY6 | pEX18 derivative carrying RDA product; Ap^{r} | This study |
| pJY8 | pEX18 derivative carrying RDA product; Ap^{r} | This study |
| pJY10 | pEX18 derivative carrying RDA product; Ap^{r} | This study |
| pJY11 | pEX18 derivative carrying RDA product; Ap^{r} | This study |
| pJY12 | pEX18 derivative carrying RDA product; Ap^{r} | This study |
| pJY13 | pEX18 derivative carrying RDA product; Ap^{r} | This study |
| pJY15 | pEX18 derivative carrying RDA product; Ap^{r} | This study |
| pJY16 | pEX18 derivative carrying RDA product; Ap^{r} | This study |
| pJY17 | pEX18 derivative carrying RDA product; Ap^{r} | This study |
| pJY19 | pEX18 derivative carrying RDA product; Ap^{r} | This study |
| pJY20 | pEX18 derivative carrying RDA product; Ap^{r} | This study |
| pJY22 | pEX18 derivative carrying RDA product; Ap^{r} | This study |
| pJY23 | pEX18 derivative carrying RDA product; Ap^{r} | This study |
| pJY24 | pEX18 derivative carrying RDA product; Ap^{r} | This study |
| pJY25 | pEX18 derivative carrying RDA product; Ap^{r} | This study |
| pJYP25 | pGEM-T Easy with a 3.0-kbp fragment | This study |
| | of pilABC gene cluster; Ap^{r} | |
| pJYPILC1-6 | pGEM-T Easy with 200-bp to 670-bp fragments | This study |
| | of pilC; Ap^{r} | |
| pJY2A | pGEM-T Easy with a 1,160-bp IPCR product | This study |
| | of pilC gene; Ap^{r} | |
| pJY5A | pGEM-T Easy with a 1,030-bp IPCR product | This study |
| | of pilA gene; Ap^{r} | |
| pJY15A | pGEM-T Easy with a 1,150-bp IPCR product | This study |
| | of uvrD gene; Ap^{r} | |
| pJY2-1 | pGEM-T Easy with a 600-bp internal | This study |
| | fragment of pilC; Ap^{r} | |
| pJY5-1 | pGEM-T Easy with a 500-bp internal | This study |
| | fragment of pilA; Ap^{r} | |
| pJY11-1 | pGEM-T Easy with a 1.3-kbp internal | This study |
| | fragment of ybtQ; Ap^{r} | |
| pJY15-1 | pGEM-T Easy with a 1.1-kbp internal | This study |
| | fragment of uvrD; Ap^{r} | |
| pJY2M | pEX18 derivative carrying a 600-bp | This study |
| | internal fragment of pilC gene; Ap^{r} | |
| pJY5M | pEX18 derivative carrying a 500-bp | This study |
| | internal fragment of pilA gene; Ap^{r} | |
| pJY11M | pEX18 derivative carrying a 1.3-kbp internal | This study |
| | fragment of ybtQ gene; Ap^{r} | |
| pJY15M | pEX18 derivative carrying a 1.1-kb internal | This study |
| | fragment of uvrD gene; Ap^{r} | |
| pJYYBT | 2.1-kb EcoRI fragment containing the intact ybtQ | This study |
| | gene cloned into pUC19 | |

Ap^{r}, ampicillin resistance;
Cb^{r}, carbenicillin resistance;
Km^{r}, kanamycin resistance;
Rif^{r}, rifampicin resistance

Molecular Genetic Techniques

Isolation of plasmid DNA, restriction enzyme digests, and agarose gel electrophoresis were performed according to standard molecular biological techniques. Plasmids were transformed into E. coli strains by standard heat shock techniques or electroporated using a Gene Pulser™ (Bio-Rad Laboratories, Richmond, Calif.) in accordance with the manufacturer's protocol. DNA restriction endonucleases and T₄ DNA ligase were used in accordance with the manufacturer's specifications. Restriction enzyme-digested chromosomal and plasmid DNA fragments were separated on 0.8% agarose gels; fragments of interest were cut from the gel under UV illumination and purified using QIAEX II Gel Extraction Kit™ (QIAGEN Inc., Valencia, Calif.).

DNA sequencing was performed at the Massachusetts General Hospital Department of Molecular Biology in the DNA Sequencing Core Facility using ABI Prism DiTerminator Cycle sequencing with AmpliTaq DNA polymerase FS and an ABI377 DNA sequencer™ (Perkin-Elmer/Applied Biosystems, Foster City, Calif.). The sequences obtained were analyzed against the P. aeruginosa PA01 genome sequence generated by the P. aeruginosa genome project (Cystic Fibrosis Foundation and Pathogenesis Corporation) and at the National Center for Biotechnology Information via the BLAST program (http://www.ncbi.nlm.nih.gov/BLAST).

Representational Difference Analysis

The procedure of RDA was originally described by Lisitsyn et al. (Science 259:946-951, 1993) and adapted to comparing bacterial strains by Tinsley and Nassif (Proc. Natl. Acad. Sci. U.S.A. 93:11109-11114, 1996) and Calia et al. (Infect. Immun. 66:849-852, 1998). In the present study, 2 μg of DNA from P. aeruginosa strain PA14 was cleaved with Sau3A1, precipitated with ethanol-sodium acetate, and ligated for 18 hours at 16° C. with 5 nmol of the oligonucleotide adapter pair (RSau24, 5′-AGCACTCTCCAGCCTCTCACCGCA-3′ (SEQ ID NO:9) and RSau12, 5′-GATCTGCGGTGA-3′ (SEQ ID NO: 10)). The mixture was gel purified on 2% low-melting-point agarose (taking fragments above 200-bp) to remove unincorporated primers, phenol purified, precipitated, and redissolved in TE buffer (10 mM Tris-HCl [pH 8.0], 1 mM EDTA) at a DNA concentration of 0.1 μg μl⁻¹. This procedure resulted in DNA fragments whose two 5′ ends are covalently linked to the 24-base adapter (SEQ ID NO:9). To prepare the subtracting DNA, chromosomal DNA of P. aeruginosa strain PA01 was sheared by repeated passage through a 30-gauge hypodermic needle to give fragments ranging from about 3- to 10-kbp. The DNA was repurified by phenol extraction, precipitated, and redissolved in TE buffer at a DNA concentration of 0.1 μg μl⁻¹. The first subtractive hybridization was performed with 40 μg of P. aeruginosa strain PA01 subtracting DNA and 400 ng of Sau3A1 digested, RSau-adapter-linked PA14 DNA fragments. The DNA was mixed, ethanol precipitated, and redissolved in 8 μl of EE buffer (10 mM N-[2-hydroxyethyl] piperazine-N′-[3-propanesulfonic acid], 1 mM EDTA [pH 8.0]). The liquid was overlaid with 30 μl of mineral oil, denatured at 100° C. for 2 minutes, and then placed at 67° C. After the addition of 2 μl of 5 M NaCl to the aqueous phase, the mixture was left to hybridize at 67° C. for 20 hours. The reaction mixture was then diluted 10-fold with preheated EE buffer-NaCl and immediately placed on ice. A portion of the subtraction mixture (10 μl) was diluted into 400 μl of PCR mix (67 mM Tris-HCl [pH 8.9], 16 mM (NH₄)₂SO₄,4 mM MgCl₂, 10 mM β-mercaptoethanol, 0.1 mg ml⁻¹ BSA, 0.125 mM each dNTP, 15 U of Taq polymerase per ml) to fill in the ends corresponding to the 24-base adapter (SEQ ID NO:9). The reaction mixture was diluted a further 10-fold, and PCR amplification was performed on 400 μl of the dilution. After denaturation for 5 minutes at 94° C. and addition of the appropriate 24-base oligonucleotide (SEQ ID NO: 9), the mixture was amplified by PCR (10 cycles of 1 minute at 95° C., 3 minutes at 72° C., with the last cycle followed by an extension at 72° C. for 10 minutes). Single-stranded DNA molecules present after amplification were degraded by a 30 minute incubation with 20 U of mung bean nuclease, diluted (1:5) in 50 mM Tris-HCl (pH 8.9), and then heated to 95° C. for 5 minutes to inactivate the enzyme. A portion (40 μl) of the solution was further amplified for 20 cycles under the same conditions as above. The amplified P. aeruginosa DNA fragments were separated by agarose gel electrophoresis from the primers and high-molecular-weight subtracting DNA. The adapters (SEQ ID NOs:9 and 10) were cleaved from the PCR products by digestion with Sau3A1, and 2 nmol of the second-round adapters (JSau24, 5′-ACCGACGTCGACTATCCATGAACA-3′ (SEQ ID NO: 11) and JSau12,5′-GATCTGTTCATG-3′(SEQ ID NO: 12)) were ligated with 2 μg of the first round difference products in a volume of 50 μl. The ligated fragments were gel purified, phenol extracted, and ethanol precipitated. A second round of subtractive hybridization/PCR enrichment was performed with 400 ng of first-round products religated to the adapters (SEQ ID NOs: 11 and 12) and 40 μg of sheared DNA from P. aeruginosa strain PA01 as above. Fragments amplified from the second round were cleaved with Sau3A1, gel purified, and cloned into the pEX18 vector (Hoang et al., Gene 212:77-86, 1998) digested with BamHI. The recombinant plasmids were maintained in E. coli strain DH5α. DNA sequences of the RDA products corresponding to the inserted DNA were determined using primers flanking the polylinker site of pEX18. The pool of fragments obtained after the second round of RDA was also tested by Southern hybridization to ensure they were absent in PA01 and present in PA14.

Southern Hybridization

Bacteria from 10 ml of LB broth were resuspended in 1 ml of 10 mM Tris-HCl (pH 8.0)-10 mM EDTA-100 mM NaCl containing 2 μg of RNase A. After addition of 50 μl of 20% sodium dodecyl sulfate and incubation at 65° C. for 30 minute, the mixture was digested for 2 hours at 37° C. with proteinase K (100 μg). The solution was then extracted once with an equal volume of phenol (pH 8.0), twice with phenol-chloroform-isopropanol (25:24:1), and once with chloroform-isopropanol (24:1). The solution was overlaid with an equal volume of ethanol and cooled to 0° C., and the DNA was pooled from the interface by mixing with a glass Pasteur pipette. DNA was washed in 70% ethanol, partially dried, and redissolved in TE buffer. The concentration of DNA was determined by UV spectrophotometry.

After digestion of purified DNA with Sau3A1 and separation by agarose gel electrophoresis, Southern blotting was performed by capillary transfer onto HYBOND-N⁺ positively charged nylon membranes (Amersham Pharmacia Biotech., Piscataway, N.J.). Hybridization of labelled probes and detection were performed with the ENHANCED CHEMILUMINESCENCE kit (Amersham) as described by the manufacturer.

Inverse PCR

To obtain chromosomal sequences flanking RDA fragments from pilC, pilA, and uvrD, partial inverse PCR (IPCR) was performed (Pang et al., BioTechniques 22:1046-1048, 1997). P. aeruginosa strain PA14 chromosomal DNA (10 μg) was partially digested with Sau3A1 at 2 U μg⁻¹ for 1 hour. The reactions were stopped by heating at 65° C. for 20 minutes and an aliquot of the reaction mixture was run on a 0.8% agarose gel to check the extent of cutting. The remaining DNA was ethanol-precipitated and resuspended in 1× ligation buffer to a concentration of 5 ng μl⁻¹. T₄ DNA ligase was added and chromosomal fragments were allowed to self-ligate at 22° C. for 4 hours. Ligation was stopped by heating to 65° C. for 20 minutes followed by phenol extraction, ethanol precipitation, and resuspension in TE buffer at a DNA concentration of 100 ng μl⁻¹. Two primers 5′-CATTTAGGGAAGCTCATCA-3′ (SEQ ID NO:13) and 5′-GAACTGTGGGACCACTTTTATC-3′ (SEQ ID NO: 14): pilC; 5′-CTAGTGAAAGGGCAGGCCT-3′ (SEQ ID NO: 15) and 5′-GGCATGCAAGATGCTTTA-3′ (SEQ ID NO: 16): pilA; 5′-ACTCTTCTTCAAGTTCGGA-3′ (SEQ ID NO: 17) and 5′-CAGATGCAGGGCAAGTTCT-3′ (SEQ ID NO:18): uvrD; facing outwards from the RDA sequences of the pilC, pilA, and uvrD genes respectively, were used to carry out each IPCR. The PCR reaction mixture (20 μl) contained 200 ng re-ligated DNA, 0.2 mM of each dNTP, 2 mM of magnesium, 1 μM of each primer, and 1 U of Taq DNA polymerase in 1× buffer supplied by the manufacturer. PCR was performed for 30 cycles of 94° C. for 1 minute, 50° C. for 1 minute, and 72° C. for 2 minutes. The largest IPCR products (SEQ ID NO: 5, pilC gene, SEQ ID NO: 3, pilA gene, and SEQ ID NO: 7, uvrD gene) were excised from a gel, purified with the QIAEX II Gel Extraction Kit (QIAGEN Inc., Valencia, Calif.), and cloned into the PCR cloning vector pGEM-T Easy, to generate pJY2A, pJY5A, and pJY15A. The ligated DNAs were used to transform competent cells of E. coli DH5α. DNA sequencing confirmed that plasmids were carrying the correct insert corresponding to each RDA product.

To confirm that the pilC fragment in pJY2A and the pilA fragment in pJY5A were located near each other in the pilABC gene cluster, a 3.0-kbp fragment of pilABC was generated by PCR using the JY2F primer (5′-TGATGAGCTTCCCTAAATG-3′ (SEQ ID NO:19); 5′ sequence of pJY2) in combination with the JY5G primer (5′-ACTGGACATAGGGGG TAAG-3′ (SEQ ID NO:20); 3′ sequence of pJY5). The amplified product was cloned into the PCR cloning vector pGEM-T Easy, and designated as pJYP25.

Cloning of the ybtQ Gene from a Plasmid Library

Chromosomal DNA of P. aeruginosa strain PA14 was fully digested with various restriction enzymes, electrophoresed, and transferred to HYBOND-N⁺ positively charged nylon membranes (Amersham). Southern blot analysis was performed as described above, using the insert of plasmid pJY11 as a probe; digestion with EcoRI gave a single hybridizing fragment of 2.1-kilobase pair. Chromosomal DNA of PA14 was digested with EcoRI, 2.0 to 3.0-kilobase pair fragments were recovered from the agarose gel after electrophoresis. The recovered fragments were then extracted from the gel with the QIAEX II Gel Extraction Kit Company. The recovered DNA fragments were ligated into pUC19, digested with EcoRI, and treated with shrimp alkaline phosphatase (Boehringer Mannheim, Indianapolis, Ind.). The resulting plasmids were transformed into ultracompetent E. coli DH5α. This library of plasmids was screened by colony blot hybridization (Sambrook et al., Molecular Cloning: A Laboratory Manual., 2^{nd} Ed., Plainview, N.Y., Cold Spring Harbor, 1989) using the insert DNA of plasmid pJY11 as a probe. Hybridization of labeled probes, detection, and washing were performed with the ENHANCED CHEMILUMINESCENCE kit as described above. Individual positive plasmid clones with appropriate insert sizes were verified by Southern blot hybridization and the DNA sequence of the 2,126-bp insert in plasmid pJYYBT was determined. DNA and deduced amino acid sequences were analyzed using CLONEMAP version 2.11 (CGC Scientific, Inc., Ballwin, Mo.) and the WU-Blast2 program in EMBL (European Bioinformatics Institute) (http://www2.ebi.ac.uk/blast2/). Motif analysis of deduced amino acid sequences was performed using the ExPASy Molecular Biology Server (http://www.expasy.ch).

Strain Constructions

P. aeruginosa strain PA14 pilC, pilA, ybtQ, and uvrD mutants JY2M, JY5M, JY11M and JY15M were constructed by inserting a suicide vector, containing an internal fragment of each gene, into the chromosome of PA14. A 700-bp internal fragment of pilC from PA14 was amplified by PCR (94° C. for 3 minutes, 25 cycles at 94° C. for 1 minute, 50° C. for 2 minutes, 72° C. for 5 minutes, 72° C. for 10 minutes), using primer pairs JY2R (5′-GCAGCAAGGTCAAAGGAGAG-3′ (SEQ ID NO:27)) and JY2L (5′-TGAGCTTCCCTAAATGCAAAAG-3′ (SEQ ID NO:28)), and cloned into the PCR cloning vector pGEM-T Easy vector to create plasmid pJY2-1. The 500-base pair, 1.3-kilobase pair and 1.1-kilobase pair internal fragments of pilA, ybtQ and uvrD genes were similarly amplified using primer pairs JY5R (5′-GAAAGGCTTTACCTTGAT-3′(SEQ ID NO:29)) and JY5L (5′-AGGAGCGAAACGAGCCG-3′(SEQ ID NO:30)), JY11R (5′-CTACGCAATCATGGCAGTA-3′(SEQ ID NO:31)) and JY11L (5′-CGATTCCATGCAGCCTGTGT-3′(SEQ ID NO:32)), JY15R (5′-CACGCATGCATTGTAGCGA-3′(SEQ ID NO:33)) and JY15L (5′-GATCGGTAGCGCAAAACT-3′(SEQ ID NO:34) respectively, and cloned into pGEM T Easy to generate plasmids pJY5-1, pJY11-1, and pJY5-1. The SacI-SphI fragments from pJY2-1, pJY5-1, pJY11-1, and pJY15-1 were cloned into the SacI and SphI sites in the polylinker of pEX18 to generate plasmids pJY2M, pJY5M, pJY11M, and pJY15M. Plasmid constructions were verified by DNA sequencing.

Plasmids pJY2M, pJY5M, pJY11M, and pJY15M were transformed into E. coli SM10 and subsequently transferred to P. aeruginosa strain PA14 by conjugation. Carbenicillin and rifampicin resistant transconjugants contained the mobilized plasmid integrated into their genomes by homologous recombination. Insertional mutation was confirmed by Southern hybridization of chromosomal DNA for each mutant strain, and compared with PA14, using the inserts of plasmids pJY2, pJY5, pJY11, and pJY15 as probes as previously described.

Virulence Testing

The virulence of various strains of P. aeruginosa was determined for a number of non-vertebrate hosts including G. mellonella (wax moth caterpillars) and C. elegans. To examine virulence in G. mellonella, overnight cultures were grown in LB broth, diluted 1:100 in the same medium, and grown to an optical density at 600 nm of 0.3 to 0.4. Cultures were pelleted and resuspended in 10 mM MgSO₄. After dilution to an optical density at 600 nm of 0.1 with 10 mM MgSO₄, serial 10-fold dilutions were made in 10 mM MgSO₄ with 2 mg of rifampicin per ml for P. aeruginosa strain PA14 (and derivatives), and 0.5 μg of ampicillin per ml for strain PA01. A 10-μl Hamilton syringe was used to inject 5-μl aliquots into individual, fifth instar G. mellonella larvae (Van der Horst Wholesale, St. Marys, Ohio), via the hindmost left proleg. A series of 10-fold serial dilutions containing from 10⁶ to 0 bacteria were injected into the G. mellonella larvae. Ten larvae were injected at each dilution and larvae were scored as live or dead after 60 hours at 25° C. Data from three independent experiments were combined. The Systat computer program was used to fit a curve to the infection data using the following formula: Y=A+(1−A)/(1+exp[B−G 1n(X)]), where X is the number of bacteria injected, Y is the fraction of larvae killed by the infection, A is the fraction of larvae killed by control injections, and B and G are parameters which are varied for optimal fit of the curve to the data points. The LD₅₀ is calculated from the curve. Statistical significance of differences was determined by using Fisher's exact test. Differences between groups were considered statistically significant if P was less than or equal to 0.05. Slow killing assays in C. elegans were performed as described previously (Tan et al., Proc. Natl. Acad. Sci. U.S. 96:2408-2413,1999).

To examine virulence of various strains in mice, a 5% total surface area burn was fashioned on the outstretched abdominal skin of 6-week-old male AKR/J mice weighing between 25 and 30 g (The Jackson Laboratories, Bar Harbor, Me.). Immediately following the burn, mice were injected subcutaneously with 5×10⁵ CFU of the P. aeruginosa strain being analyzed, and the number of animals that died as a result of sepsis was monitored each day for 10 days. For each strain, data from two independent experiments (eight mice per each experiment) were combined (except that P. aeruginosa strain PA01 was tested only once). Animal study protocols were reviewed and approved by the Institutional Animal Care and Use Committee.

Isolation of Additional Virulence Genes

Based on the nucleotide and amino acid sequences (FIGS. 3, 6, 7, and 8) described herein, the isolation of additional sequences of virulence factors is made possible using standard strategies and techniques that are well known in the art. Any pathogenic cell can serve as a nucleic acid source for the molecular cloning of such virulence genes. Exemplary pathogenic bacteria include, without limitation, Aerobacter, Aeromonas, Acinetobacter, Agrobacterium, Bacillus, Bacteroides, Bartonella, Bortella, Brucella, Calymmatobacterium, Campylobacter, Citrobacter, Clostridium, Cornyebacterium, Enterobacter, Escherichia, Francisella, Haemophilus, Hafnia, Helicobacter, Klebsiella, Legionella, Listeria, Morganella, Moraxella, Proteus, Providencia, Pseudomonas, Salmonella, Serratia, Shigella, Staphylococcus, Streptococcus, Treponema, Xanthomonas, Vibrio, and Yersinia.

In one particular example of such an isolation technique, any one of the nucleotide sequences described herein may be used, together with conventional screening methods of nucleic acid hybridization screening. Such hybridization techniques and screening procedures are well known to those skilled in the art and are described, for example, in Benton and Davis (Science 196:180-182, 1977); Grunstein and Hogness (Proc. Natl. Acad. Sci., USA 72:3961-3965, 1975); Ausubel et al. (Current Protocols in Molecular Biology, Wiley Interscience, New York, 1997); Berger and Kimmel (Berger and Kimmel, Guide to Molecular Cloning Techniques, 1987, Academic Press, New York); and Sambrook et al., (Molecular Cloning: A Laboratory Manual, Cold Spring Harbor Laboratory Press, New York). In one particular example, all or part of the ybtQ sequence (described herein) may be used as a probe to screen a recombinant bacterial DNA library for genes having sequence identity to the ybtQ gene. Hybridizing sequences are then detected by plaque or colony hybridization according to standard methods.

Alternatively, using all or a portion of the amino acid sequence of the YbtQ polypeptide, one may readily design YbtQ-specific oligonucleotide probes, including degenerate oligonucleotide probes (i.e., a mixture of all possible coding sequences for a given amino acid sequence). These oligonucleotides may be based upon the sequence of either DNA strand and any appropriate portion of the ybtQ sequence (SEQ ID NO:1) of the YbtQ protein. General methods for designing and preparing such probes are provided, for example, in Ausubel et al. (supra), and Berger and Kimmel (Guide to Molecular Cloning Techniques, 1987, Academic Press, New York). These oligonucleotides are useful for ybtQ gene isolation, either through their use as probes capable of hybridizing to ybtQ complementary sequences or as primers for various amplification techniques, for example, polymerase chain reaction (PCR) cloning strategies. If desired, a combination of different, detectably-labeled oligonucleotide probes may be used for the screening of a recombinant DNA library. Such libraries are prepared according to methods well known in the art, for example, as described in Ausubel et al. (supra), or they may be obtained from commercial sources.

As discussed above, sequence-specific oligonucleotides may also be used as primers in amplification cloning strategies, for example, using PCR. PCR methods are well known in the art and are described, for example, in PCR Technology, Erlich, ed., Stockton Press, London, 1989; PCR Protocols: A Guide to Methods and Applications, Innis et al., eds., Academic Press, Inc., New York, 1990; and Ausubel et al. (supra). Primers are optionally designed to allow cloning of the amplified product into a suitable vector, for example, by including appropriate restriction sites at the 5′ and 3′ ends of the amplified fragment (as described herein). If desired, nucleotide sequences may be isolated using the PCR “RACE” technique, or Rapid Amplification of cDNA Ends (see, e.g., Innis et al. (supra)). By this method, oligonucleotide primers based on a desired sequence are oriented in the 3′ and 5′ directions and are used to generate overlapping PCR fragments. These overlapping 3′- and 5′-end RACE products are combined to produce an intact full-length cDNA. This method is described in, for example, Innis et al. (supra); and Frohman et al., Proc. Natl. Acad. Sci. USA 85:8998-9002, 1988.

Partial virulence sequences, e.g., sequence tags, are also useful as hybridization probes for identifying full-length sequences, as well as for screening databases for identifying previously unidentified related virulence genes. For example, as is described above, the sequences of pJY2, pJY5, and pJY15 were expanded to those encompassed by pJY2A, pJY5A, and pJY15A, respectively.

Confirmation of a sequence's relatedness to a virulence polypeptide may be accomplished by a variety of conventional methods including, but not limited to, functional complementation assays and sequence comparison of the gene and its expressed product. In addition, the activity of the gene product may be evaluated according to any of the techniques described herein, for example, the functional or immunological properties of its encoded product. Alternatively, the gene product may be evaluated in a plant model such as Arabidopsis or an animal model, such as G. mellonella, C. elegans, or the mouse-burn assay using methods known in the art.

Once an appropriate sequence is identified, it is cloned according to standard methods and may be used, for example, for screening compounds that reduce the virulence of a pathogen.

Polypeptide Expression

In general, polypeptides of the invention may be produced by transformation of a suitable host cell with all or part of a polypeptide-encoding nucleic acid molecule or fragment thereof in a suitable expression vehicle.

Those skilled in the field of molecular biology will understand that any of a wide variety of expression systems may be used to provide the recombinant protein. The precise host cell used is not critical to the invention. A polypeptide of the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (Novagen, Inc., Madison, Wis. According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Once the recombinant polypeptide of the invention is expressed, it is isolated, e.g., using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra).

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980).

Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.).

These general techniques of polypeptide expression and purification can also be used to produce and isolate useful peptide fragments or analogs (described herein).

Antibodies

To generate antibodies, a coding sequence for a polypeptide of the invention may be expressed as a C-terminal fusion with glutathione S-transferase (GST) (Smith et al., Gene 67:31-40, 1988). The fusion protein is purified on glutathione-Sepharose beads, eluted with glutathione, cleaved with thrombin (at the engineered cleavage site), and purified to the degree necessary for immunization of rabbits. Primary immunizations, for example, are carried out with Freund's complete adjuvant and subsequent immunizations with Freund's incomplete adjuvant. Antibody titers are monitored by Western blot and immunoprecipitation analyses using the thrombin-cleaved protein fragment of the GST fusion protein. Immune sera are affinity purified using CNBr-Sepharose-coupled protein. Antiserum specificity is determined using a panel of unrelated GST proteins.

As an alternate or adjunct immunogen to GST fusion proteins, peptides corresponding to relatively unique immunogenic regions of a polypeptide of the invention may be generated and coupled to keyhole limpet hemocyanin (KLH) through an introduced C-terminal lysine. Antiserum to each of these peptides is similarly affinity purified on peptides conjugated to BSA, and specificity tested in ELISA and Western blots using peptide conjugates, and by Western blot and immunoprecipitation using the polypeptide expressed as a GST fusion protein.

Alternatively, monoclonal antibodies which specifically bind any one of the polypeptides of the invention are prepared according to standard hybridoma technology (see, e.g., Kohler et al., Nature 256:495-497, 1975; Kohler et al., Eur. J. Immunol. 6:511-519, 1976; Kohler et al., Eur. J. Immunol 6:292-295, 1976; Hammerling et al., In Monoclonal Antibodies and T Cell Hybridomas, Elsevier, N.Y., 1981; Ausubel et al., supra). Once produced, monoclonal antibodies are also tested for specific recognition by Western blot or immunoprecipitation analysis (by the methods described in Ausubel et al., supra). Antibodies that specifically recognize the polypeptide of the invention are considered to be useful in the invention; such antibodies may be used, e.g., in an immunoassay. Alternatively monoclonal antibodies may be prepared using the polypeptide of the invention described above and a phage display library (Vaughan et al., Nature Biotech 14:309-314, 1996).

Preferably, antibodies of the invention are produced using fragments of the polypeptide of the invention that lie outside generally conserved regions and appear likely to be antigenic, by criteria such as high frequency of charged residues. In one specific example, such fragments are generated by standard techniques of PCR and cloned into the pGEX expression vector (Ausubel et al., supra). Fusion proteins are expressed in E. coli and purified using a glutathione agarose affinity matrix as described in Ausubel et al. (supra). To attempt to minimize the potential problems of low affinity or specificity of antisera, two or three such fusions are generated for each protein, and each fusion is injected into at least two rabbits. Antisera are raised by injections in a series, preferably including at least three booster injections.

Diagnostics

In addition, PilA, PilC, UvrD, and YbtQ proteins may be used in diagnosing a Pseudomonas aeruginosa infection in an organism, where the presence of PilA, PilC, UvrD, or YbtQ proteins or nucleic acids may provide an indication of an infection. PilA, PilC, UvrD, or YbtQ protein or nucleic acid expression may be assayed by any standard technique. For example, polypeptide expression in a biological sample may be monitored by standard Northern blot analysis, using, for example, probes designed from a pilA, pilC, uvrD, or ybtQ nucleic acid sequences, or from nucleic acid sequences that hybridize to a pilA, pilC, uvrD, or ybtQ nucleic acid sequence. Measurement of such expression may be aided by PCR (see, e.g., Ausubel et al., Current Protocols in Molecular Biology, Wiley Interscience, New York, 2000; PCR Technology: Principles and Applications for DNA Amplification, ed., H. A. Ehrlich, Stockton Press, NY; and Yap and McGee, Nucl. Acids Res. 19:4294, 1991).

In yet another approach, immunoassays may be used to detect or monitor a PilA, PilC, UvrD, or YbtQ polypeptide in a biological sample. PilA, PilC, UvrD, or YbtQ specific polyclonal or monoclonal antibodies may be used in any standard immunoassay format (e.g., ELISA, Western blot, or RIA assay) to measure PilA, PilC, UvrD, and YbtQ polypeptide levels; the presence of a PilA, PilC, UvrD, or YbtQ polypeptide is taken as an indication of a bacterial infection. Examples of immunoassays are described, e.g., in Ausubel et al. (Current Protocols in Molecular Biology, John Wiley & Sons, New York, 2000). Immunohistochemical techniques may also be utilized for PilA, PilC, UvrD, or YbtQ detection. For example, a sample may be obtained from a patient, and that sample stained for the presence of a PilA, PilC, UvrD, or YbtQ protein using an antibody against that protein and any standard detection system (e.g., one which includes a secondary antibody conjugated to horseradish peroxidase). General guidance regarding such techniques can be found in, e.g., Bancroft and Stevens (Theory and Practice of Histological Techniques, Churchill Livingstone, 1982) and Ausubel et al. (Current Protocols in Molecular Biology, John Wiley & Sons, New York, 2000).

The PilA, PilC, UvrD, or YbtQ diagnostic assays described above may be carried out using any biological sample (for example, any bodily fluid, such as sputum) in which pilA, pilC, uvrD, or ybtQ nucleic acids or proteins are expressed during infection.

Screening Assays

As discussed above, a number of P. aeruginosa virulence factors were identified that are involved in pathogenicity, and that may therefore be used to screen for compounds that reduce the virulence of that organism, as well as other microbial pathogens. Any number of methods are available for carrying out such screening assays. According to one approach, candidate compounds are added at varying concentrations to the culture medium of pathogenic cells expressing one of the nucleic acid sequences of the invention. Gene expression is then measured, for example, by standard Northern blot analysis (Ausubel et al., Current Protocols ill Molecular Biology, Wiley Interscience, New York, 2000), using any appropriate fragment prepared from the nucleic acid molecule as a hybridization probe. The level of gene expression in the presence of the candidate compound is compared to the level measured in a control culture medium lacking the candidate molecule. A compound that promotes a decrease in the expression of the virulence factor is considered useful in the invention; such a molecule may be used, for example, as a therapeutic to combat the pathogenicity of an infectious organism.

If desired, the effect of candidate compounds may, in the alternative, be measured at the level of polypeptide production using the same general approach and standard immunological techniques, such as Western blotting or immunoprecipitation with an antibody specific for a pathogenicity factor. For example, immunoassays may be used to detect or monitor the expression of at least one of the polypeptides of the invention in a pathogenic organism. Polyclonal or monoclonal antibodies (produced as described above) that are capable of binding to such a polypeptide may be used in any standard immunoassay format (e.g., ELISA, Western blot, or RIA assay) to measure the level of the pathogenicity polypeptide. A compound that promotes a decrease in the expression of the pathogenicity polypeptide is considered particularly useful. Again, such a molecule may be used, for example, as a therapeutic to combat the pathogenicity of an infectious organism.

Alternatively, or in addition, candidate compounds may be identified that specifically bind to and inhibit a pathogenicity polypeptide of the invention. The efficacy of such a candidate compound is dependent upon its ability to interact with the pathogenicity polypeptide. Such an interaction can be readily assayed using any number of standard binding techniques and functional assays (e.g., those described in Ausubel et al., supra). For example, a candidate compound may be tested int vitro for interaction and binding with a polypeptide of the invention and its ability to modulate pathogenicity may be assayed by any standard assays (e.g., those described herein).

Potential antagonists include organic molecules, peptides, peptide mimetics, polypeptides, nucleic acid ligands, and antibodies that bind to a nucleic acid sequence or polypeptide of the invention and thereby inhibit or extinguish its activity. Potential antagonists also include small molecules that bind to and occupy the binding site of the polypeptide thereby preventing binding to cellular binding molecules, such that normal biological activity is prevented. Other potential antagonists include antisense molecules.

In one particular example, a candidate compound that binds to a pathogenicity polypeptide may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the pathogenicity polypeptide is identified on the basis of its ability to bind to the pathogenicity polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). In addition, these candidate compounds may be tested for their ability to render a pathogen less virulent (e.g., as described herein). Compounds isolated by this approach may also be used, for example, as therapeutics to treat or prevent the onset of a pathogenic infection, disease, or both. Compounds that are identified as binding to pathogenicity polypeptides with an affinity constant less than or equal to 10 mM are considered particularly useful in the invention.

In yet another approach, candidate compounds are screened for the ability to inhibit the virulence of a Pseudomonas cell by monitoring the effect of the compound on the production of a phenolate-thiazole siderophore. According to one approach, candidate compounds are added at varying concentrations to a culture medium of pathogenic cells. A phenolate-thiazole siderophore is then measured according to any standard method. The level of phenolate-thiazole siderophore production in the presence of the candidate compound is compared to the level measured in a control culture lacking the candidate molecule. A compound that promotes a decrease in the expression of the siderophore is considered useful in the invention; such a molecule may be used, for example, as a therapeutic to combat the pathogenicity of an infectious organism. Optionally, compounds identified in any of the above-described assays may be confirmed as useful in conferring protection against the development of a pathogenic infection in any standard animal model (e.g., the mouse-burn assay described herein) and, if successful, may be used as anti-pathogen therapeutics.

Each of the DNA sequences provided herein may also be used in the discovery and development of antipathogenic compounds (e.g., antibiotics). The encoded protein, upon expression, can be used as a target for the screening of antibacterial drugs. Additionally, the DNA sequences encoding the amino terminal regions of the encoded protein or Shine-Delgarno or other translation facilitating sequences of the respective mRNA can be used to construct antisense sequences to control the expression of the coding sequence of interest.

The invention also provides the use of the polypeptide, polynucleotide, or inhibitor of the invention to interfere with the initial physical interaction between a pathogen and mammalian host responsible for infection. In particular the molecules of the invention may be used: in the prevention of adhesion and colonization of bacteria to mammalian extracellular matrix proteins; to extracellular matrix proteins in wounds; to block mammalian cell invasion; or to block the normal progression of pathogenesis.

The antagonists and agonists of the invention may be employed, for instance, to inhibit and treat a variety of bacterial infections.

Optionally, compounds identified in any of the above-described assays may be confirmed as useful in conferring protection against the development of a pathogenic infection in any standard animal model (e.g., the mouse-burn assay described herein) and, if successful, may be used as anti-pathogen therapeutics (e.g, antibiotics).

Test Compounds and Extracts

In general, compounds capable of reducing pathogenic virulence are identified from large libraries of either natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-pathogenic activity should be employed whenever possible.

When a crude extract is found to have an anti-pathogenic or anti-virulence activity, or a binding activity, further fractionation of the positive lead extract is necessary to isolate chemical constituents responsible for the observed effect. Thus, the goal of the extraction, fractionation, and purification process is the careful characterization and identification of a chemical entity within the crude extract having anti-pathogenic activity. Methods of fractionation and purification of such heterogenous extracts are known in the art. If desired, compounds shown to be useful agents for the treatment of pathogenicity are chemically modified according to methods known in the art.

Pharmaceutical Therapeutics and Plant Protectants

The invention provides a simple means for identifying compounds (including peptides, small molecule inhibitors, and mimetics) capable of inhibiting the pathogenicity or virulence of a pathogen. Accordingly, a chemical entity discovered to have medicinal or agricultural value using the methods described herein are useful as either drugs, plant protectants, or as information for structural modification of existing anti-pathogenic compounds, e.g., by rational drug design.

For therapeutic uses, the compositions or agents identified using the methods disclosed herein may be administered systemically, for example, formulated in a pharmaceutically-acceptable buffer such as physiological saline. Treatment may be accomplished directly, e.g., by treating the animal with antagonists which disrupt, suppress, attenuate, or neutralize the biological events associated with a pathogenicity polypeptide. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of an anti-pathogenic agent in a physiologically acceptable carrier. Suitable carriers and their formulation are described, for example, in Remington's Pharmaceutical Sciences by E. W. Martin. The amount of the anti-pathogenic agent to be administered varies depending upon the manner of administration, the age and body weight of the patient, and with the type of disease and extensiveness of the disease. Generally, amounts will be in the range of those used for other agents used in the treatment of other microbial diseases, although in certain instances lower amounts will be needed because of the increased specificity of the compound. A compound is administered at a dosage that inhibits microbial proliferation. For example, for systemic administration a compound is administered typically in the range of 0.1 ng-10 g/kg body weight.

For agricultural uses, the compositions or agents identified using the methods disclosed herein may be used as chemicals applied as sprays or dusts on the foliage of plants. Typically, such agents are to be administered on the surface of the plant in advance of the pathogen in order to prevent infection. Seeds, bulbs, roots, tubers, and corms are also treated to prevent pathogenic attack after planting by controlling pathogens carried on them or existing in the soil at the planting site. Soil to be planted with vegetables, ornamentals, shrubs, or trees can also be treated with chemical fumigants for control of a variety of microbial pathogens. Treatment is preferably done several days or weeks before planting. The chemicals can be applied by either a mechanized route, e.g., a tractor or with hand applications. In addition, chemicals identified using the methods of the assay can be used as disinfectants.

Vaccine Production

The invention also provides for a method of inducing an immunological response in an individual, particularly a human, which comprises inoculating the individual with the polypeptides of the invention, or fragments thereof, in a suitable carrier for the purpose of inducing an immune response to protect said individual from infection, particularly bacterial infection, and most particularly Pseudomonas aeruginosa PA14 infection. The administration of this immunological composition may be used either therapeutically in individuals already experiencing bacterial infection, or may be used prophylactically to prevent bacterial infection.

The preparation of vaccines that contain immunogenic polypeptides is known to one skilled in the art. The polypeptide may serve as an antigen for vaccination, or an expression vector encoding the polypeptide, or fragments or variants thereof, may be delivered in vivo in order to induce an immunological response comprising the production of antibodies or a T cell immune response.

For example, the YbtQ, PilA, or PilC polypeptides, or fragments or variants thereof might be delivered in vivo in order to induce an immune response. The polypeptides might be fused to a recombinant protein that stabilizes the polypeptide of the invention, aids in its solubilization, facilitates its production or purification, or acts as an adjuvant by providing additional stimulation of the immune system. The compositions and methods comprising the polypeptides or nucleotides of the invention and immunostimulatory DNA sequences are described in Sato et al., (Science 273:352-354, 1996).

Typically vaccines are prepared in an injectable form, either as a liquid solution or as a suspension. Solid forms suitable for injection may also be prepared as emulsions, or with the polypeptides encapsulated in liposomes. Vaccine antigens are usually combined with a pharmaceutically acceptable carrier, which includes any carrier that does not induce the production of antibodies harmful to the individual receiving the carrier. Suitable carriers typically comprise large macromolecules that are slowly metabolized, such as proteins, polysaccharides, polylactic acids, polyglycolic acids, polymeric amino acids, amino acid copolymers, lipid aggregates, and inactive virus particles. Such carriers are well known to those skilled in the art. These carriers may also function as adjuvants.

Adjuvants are immunostimulating agents that enhance vaccine effectiveness. Effective adjuvants include, but are not limited to, aluminum salts such as aluminum hydroxide and aluminum phosphate, muramyl peptides, bacterial cell wall components, saponin adjuvants, and other substances that act as immunostimulating agents to enhance the effectiveness of the composition.

Immunogenic compositions, i.e. the antigen, pharmaceutically acceptable carrier and adjuvant, also typically contain diluents, such as water, saline, glycerol, ethanol. Auxiliary substances may also be present, such as wetting or emulsifying agents, pH buffering substances, and the like. Proteins may be formulated into the vaccine as neutral or salt forms. The vaccines are typically administered parenterally, by injection; such injection may be either subcutaneously or intramuscularly. Additional formulations are suitable for other forms of administration, such as by suppository or orally. Oral compositions may be administered as a solution, suspension, tablet, pill, capsule, or sustained release formulation.

In addition, the vaccine can also be administered to individuals to generate polyclonal antibodies (purified or isolated from serum using standard methods) that may be used to passively immunize an individual. These polyclonal antibodies can also serve as immunochemical reagents.

In addition, it is possible to prepare live attenuated microorganism vaccines that express recombinant polypeptides, for example of the YbtQ, PilA or PilC antigens. Suitable attenuated microorganisms are known in the art, and include, for example, viruses and bacteria.

Vaccines are administered in a manner compatible with the dose formulation. The immunogenic composition of the vaccine comprises an immunologically effective amount of the antigenic polypeptides and other previously mentioned components. By an immunologically effective amount is meant a single dose, or a vaccine administered in a multiple dose schedule, that is effective for the treatment or prevention of an infection. The dose administered will vary, depending on the subject to be treated, the subject's health and physical condition, the capacity of the subject's immune system to produce antibodies, the degree of protection desired, and other relevant factors. Precise amounts of the active ingredient required will depend on the judgement of the practitioner, but typically range between 5 μg to 250 μg of antigen per dose.

All publications mentioned in this specification are herein incorporated by reference to the same extent as if each independent publication was specifically and individually indicated to be incorporated by reference.

### Other Embodiments

In general, the invention includes any nucleic acid sequence which may be isolated as described herein or which is readily isolated by homology screening or PCR amplification using the nucleic acid sequences of the invention. Also included in the invention are polypeptides which are modified in ways which do not abolish their virulence (assayed, for example as described herein). Such changes may include certain mutations, deletions, insertions, or post-translational modifications, or may involve the inclusion of any of the polypeptides of the invention as one component of a larger fusion protein. Also, included in the invention are polypeptides that have lost their virulence.

Thus, in other embodiments, the invention includes any protein which is substantially identical to a polypeptide of the invention. Such homologs include other substantially pure naturally-occurring polypeptides as well as allelic variants; natural mutants; induced mutants; proteins encoded by DNA that hybridizes to any one of the nucleic acid sequences of the invention under high stringency conditions or, less preferably, under low stringency conditions (e.g., washing at 2×SSC at 40° C. with a probe length of at least 40 nucleotides); and proteins specifically bound by antisera of the invention.

The invention further includes analogs of any naturally-occurring polypeptide of the invention. Analogs can differ from the naturally-occurring the polypeptide of the invention by amino acid sequence differences, by post-translational modifications, or by both. Analogs of the invention will generally exhibit at least 85%, more preferably 90%, and most preferably 95% or even 99% identity with all or part of a naturally-occurring amino, acid sequence of the invention. The length of sequence comparison is at least 15 amino acid residues, preferably at least 25 amino acid residues, and more preferably more than 35 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the invention also includes fragments of any one of the polypeptides of the invention. As used herein, the term “a fragment” means at least 5, preferably at least 20 contiguous amino acids, preferably at least 30 contiguous amino acids, more preferably at least 50 contiguous amino acids, and most preferably at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

Furthermore, the invention includes nucleotide sequences that facilitate specific detection of any of the nucleic acid sequences of the invention. Thus, for example, nucleic acid sequences described herein or fragments thereof may be used as probes to hybridize to nucleotide sequences by standard hybridization techniques under conventional conditions. Sequences that hybridize to a nucleic acid sequence coding sequence or its complement are considered useful in the invention. Sequences that hybridize to a coding sequence of a nucleic acid sequence of the invention or its complement and that encode a polypeptide of the invention are also considered useful in the invention. As used herein, the term “fragment,” as applied to nucleic acid sequences, means at least 5 contiguous nucleotides, preferably at least 10 contiguous nucleotides, more preferably at least 20 to 30 contiguous nucleotides, and most preferably at least 40 to 80 or more contiguous nucleotides. Fragments of nucleic acid sequences can be generated by methods known to those skilled in the art.

The invention further provides a method for inducing an immunological response in an individual, particularly a human, which includes inoculating the individual with, for example, any of the polypeptides (or a fragment or analog thereof or fusion protein) of the invention to produce an antibody and/or a T cell immune response to protect the individual from infection, especially bacterial infection (e.g., a Pseudomonas aeruginosa PA14 infection). The invention further includes a method of inducing an immunological response in an individual which includes delivering to the individual a nucleic acid vector to direct the expression of a polypeptide described herein (or a fragment or fusion thereof) in order to induce an immunological response. The invention also includes vaccine compositions including the polypeptides or nucleic acid sequences of the invention. For example, the polypeptides of the invention may be used as an antigen for vaccination of a host to produce specific antibodies which protect against invasion of bacteria, for example, by blocking the production of a phenolate-thiazole siderophore. The invention therefore includes a vaccine formulation which includes an immunogenic recombinant polypeptide of the invention together with a suitable carrier.

All publications and patent applications mentioned in this specification are herein incorporated by reference to the same extent as if each independent publication or patent application was specifically and individually indicated to be incorporated by reference.

Other embodiments are within the scope of the claims.

1. An isolated polypeptide comprising an amino acid sequence having at least 30% identity to the amino acid sequence of YbtQ (SEQ ID NO:2), wherein expression of said polypeptide in a microorganism affects the virulence of said microorganism.

2. The isolated polypeptide of claim 1, said polypeptide comprising the amino acid sequence of YbtQ (SEQ ID NO:2).

3. The isolated polypeptide of claim 1, wherein said polyppetide consists consists essentially of the amino acid sequence of YbtQ (SEQ ID NO:2) or a fragment thereof.

4. An isolated polypeptide fragment of the isolated polypeptide of claim 1.

5. An isolated nucleic acid molecule having at least 30% identity to the nucleotide sequence of ybtQ (SEQ ID NO:1) encoding the YbtQ polypeptide (SEQ ID NO:2), wherein expression of said nucleic acid molecule, in a microorganism, affects the virulence of said microorganism.

6. The isolated nucleic acid molecule of claim 5, said nucleic acid molecule comprising the nucleotide sequence of ybtQ (SEQ ID NO:1) or a complement thereof.

7. The isolated nucleic acid molecule of claim 5, said nucleic acid molecule consisting essentially of the nucleotide sequence of ybtQ (SEQ ID NO:1) encoding the YbtQ polypeptide (SEQ ID NO:2) or a fragment thereof.

8. A vector comprising the isolated nucleic acid molecule of any one of claims 5, 6, or 7.

9. A host cell comprising the vector of claim 8.

10. A method for identifying a compound which is capable of decreasing the expression of a pathogenic virulence factor, said method comprising the steps of:
(a) providing a pathogenic cell expressing the isolated nucleic acid molecule of claim 5; and
(b) contacting said pathogenic cell with a candidate compound, a decrease in expression of said nucleic acid molecule following contact with said candidate compound identifying a compound which decreases the expression of a pathogenic virulence factor.

11. The method of claim 10, wherein said pathogenic cell infects a mammal.

12. A method for identifying a compound which binds a polypeptide, said method comprising the steps of:
(a) contacting a candidate compound with the isolated polypeptide of claim 1 under conditions that allow binding; and
(b) detecting binding of the candidate compound to the polypeptide.

13. A method of treating a pathogenic infection in mammal, said method comprising the steps of:
(a) identifying a mammal having a pathogenic infection; and
(b) administering to said mammal a therapeutically effective amount of a composition which inhibits the expression or activity of a polypeptide encoded by the isolated nucleic acid molecule of claim 5 in said pathogen.

14. The method of claim 13, wherein said pathogen is Pseudomonas aeruginosa.

15. A method of treating a pathogenic infection in a mammal, said method comprising the steps of:
(a) identifying a mammal having a pathogenic infection; and
(b) administering to said mammal a therapeutically effective amount of a composition which binds and inhibits the isolated polypeptide of claim 1.

16. The method of claim 15, wherein said pathogen is Pseudomonas aeruginosa.

17. A method of producing a polypeptide, said method comprising the steps of:
(a) providing a cell transformed with the isolated nucleic acid molecule of claim 5, 6, or 7 positioned for expression in the cell;
(b) culturing the cell under conditions for expressing the nucleic acid molecule;
(c) and isolating the polypeptide.

18. An antibody which specifically binds to the isolated polypeptide of claim 1.

19. An agonist or antagonist to the isolated polypeptide of claim 1.
